(19) United States
(12) Patent Application Publication
(10) Pub. No.: US 2026/0367067 A1
(43) Pub. Date: Nov. 19, 2026

(54) FET-BASED NANOPORE SENSING DEVICE

(71) Applicants: IMEC VZW, Leuven (BE); KATHOLIEKE UNIVERSITEIT LEUVEN, Leuven (BE)

(72) Inventors: Koen MARTENS, Leuven (BE); Sybren SANTERMANS, Leuven (BE); Lijun LIU, Leuven (BE); Pol VAN DORPE, Leuven (BE); Benjamin JONES, Leuven (BE)

(21) Appl. No.: 19/144,046

(22) PCT Filed: Dec. 21, 2023
(86) PCT No.: PCT/EP2023/087237

§ 371 Date: Jun. 27, 2025

(30) Foreign Application Priority Data
Dec. 28, 2022 (EP) 22216973.2

(51) Int. Cl. G01N 33/487; B82Y 15/00

(52) U.S. Cl. CPC G01N 33/48721; B82Y 15/00

(57) ABSTRACT

The current disclosure relates to molecule sensing using pore field effect transistor (FET) devices. More specifically, the proposed techniques relate to pore (FET) device realizing large bandwidths, high signal strengths and low noise when sensing molecules, which is attained by the unique properties of the device, such as having a wide lumen part of the pore to allow for a sufficiently large electrode, and a narrow aperture part. The disclosure also relates to systems for use thereof, and simulation methods for designing such devices.

### CROSS-REFERENCE TO RELATED APPLICATION(S)

The present application is a U.S. National Phase Application pursuant to 35 U.S.C. § 371 of International Application No. PCT/EP2023/087237, filed Dec. 21, 2023, which claims priority to European Patent Application No. 22216973.2 filed Dec. 28, 2022. The entire contents of these applications are herewith incorporated by reference into the present application.

### TECHNICAL FIELD

The present disclosure relates to molecule sensing using nanoscale pores or nanopores. More specifically, the proposed techniques relate to a field effect transistor (FET) device for sensing of target molecules, and systems for use thereof.

### BACKGROUND

Nanopore sensing has been used for DNA and RNA detection and sequencing for some time, and the interest of the research community has now also turned to nanopore sensing of biomolecules such as proteins and peptides. Nanopore sensing is attractive as it has the potential to detect multiple distinct types of biomolecules, such as DNA, RNA, and proteins, simultaneously in a complex biological sample, and since it can be integrated into small and portable devices.

Recently, there has been increasing interest in bringing together both field-effect transistors (FETs) and nanopores to develop pore FETs to sense target molecules. This can include sensing of particles, such as sensing of single molecules. The pore is used for gating the FET such that when a single molecule is passed through the pore, a current modulation of the FET can be achieved. Thus, a pore FET sensor can be formed, wherein translocation of a single molecule through the pore can affect gating of the FET for allowing detection of the single molecule. However, these devices suffer from noise, especially when applied to larger bandwidths. Accordingly, improved nanopore-FET devices for sensing of target molecules are needed.

### SUMMARY

An object of the present disclosure is to provide devices and systems, which provide high translocation signal magnitudes, high bandwidths, while suppressing short channel effects and reducing noise.

In the first aspect, the present disclosure relates to a pore Field-effect transistor (FET) device for sensing target molecules in an electrolyte. The device comprises a pore with at least a first and a second opening, consisting of two contiguous parts: a lumen part and an aperture part. The lumen part is connected to the first opening, while the aperture part is connected to the second opening. The device also includes at least a first and a second reservoir, with the pore fluidically connected to both reservoirs. The first reservoir is connected to the lumen part via the first opening, and the second reservoir is connected to the aperture part via the second opening. Additionally, the device features a sensor region comprising a main electrode that overlaps either the lumen part or the aperture part of the pore, allowing target molecules passing through the pore to pass through the sensor region. The transverse cross-section of the pore is defined by an area (A) defined by A=πr² and an effective cross-section diameter (2r), where r is the effective cross-section radius of the transverse cross-section. The geometry of the device is such that: i) the effective cross-section diameter of every transverse cross-section of the pore is less than 1 μm; ii) the aperture part has all its transverse cross-sections having an effective diameter (d_{A}) less than 50 nm; iii) an area of a transverse cross-section in the aperture part (A_{A}) is smaller than an area of a transverse cross-section in the lumen part (A_{L}), with at least ¾ of the transverse cross-sections of the lumen part having an area at least four times larger than any aperture transverse cross-section (A_{L}≥4A_{A}); and iv) the main electrode area exposed to the electrolyte is larger than 10000 nm². For instance, the objective may be obtained by a nanoscale pore Field-effect transistor (FET) device for sensing of target molecules in an electrolyte, the device comprising: a nanoscale pore having at least a first and a second opening and comprising two contiguous parts, a lumen part and an aperture part, such that the lumen part is connected at least to the first opening and the aperture part is connected to the second opening; a pore axis oriented from the first opening to the second opening of the nanoscale pore, wherein the lumen part and the aperture part of the nanoscale pore are oriented along the pore axis; at least a first and a second reservoir, the nanoscale pore being fluidically connected to the first and second reservoir, wherein the first reservoir is in connection with the lumen part of the nanoscale pore via the first opening, and the second reservoir is in connection with the aperture part of the nanoscale pore via the second opening, an electrical center or sensor region comprising a main electrode located between, and potentially overlapping, the lumen part and the aperture part of the nanoscale pore; wherein a cross-section of the nanoscale pore orthogonal to the pore axis has an Area which defines the effective cross-section radius r=\sqrt{A/π} and an effective cross-section diameter defined by d=2r, and wherein a geometry of the device is such that: i) an effective diameter of any part of the nanoscale pore is less than 1 μm; ii) the aperture part inside the pore has an effective cross-section diameter, d_{A}, less than 50 nm; iii) an area of a cross section in the aperture part, A_{A}, is smaller than an area of a cross section in the lumen, A_{L}, such that A_{L}>2A_{A}, and wherein at least ¾ of the cross sections of the lumen part have an area which is at least four times larger than the area of any aperture cross section, such that they fulfil the requirement A_{L}>4A_{A}, iv) the cross section area at each opening of the nanoscale pore, A_{O}, shows an enlargement of the pore compared to an area inside the pore, A_{I}, such that A_{O}>4A_{I}. The electrode area A_{E}>10000 nm².

In embodiments, the geometry of the device may provide that, on condition that the pore and the at least first and second reservoirs are uniformly filled with a single isotropic and uniformly conducting test material or test liquid, and that the main electrode makes electrical contact with the test material or test liquid with negligible contact resistance and negligible electrode resistance, the resistance between the aperture reservoir and the main electrode (R1) corresponds to the resistance between the lumen reservoir and the main electrode (R2), with R1/R2 ranging from ⅙ to 6.

In embodiments, the geometry of the device may provide that, on condition that the pore and the at least first and second reservoirs are uniformly filled with a single isotropic and uniformly conducting test material or test liquid, and that the main electrode makes electrical contact with the test material or test liquid with negligible contact resistance and negligible electrode resistance, R_{A}/R_{L} is from 0.27 to 23, such as from 0.57 to 8.6, 0.80 to 5.0, 1.00 to 3.00, or 2, wherein R_{A} is the test resistance of the aperture part, which is the electrical resistance between the main electrode and the second reservoir, which is fluidically connected to the aperture, wherein R_{L} is the test resistance of the lumen part, which is the resistance between the main electrode and the reservoir or reservoirs fluidically connected to the lumen part of the pore via the first opening. In embodiments, the geometry of the device may provide that, on condition that the pore (4) and the at least first and second reservoirs are each uniformly filled with a different single isotropic and uniformly conducting test material or test liquid, wherein each reservoir connected to the lumen part of the pore via the first opening has a test material or test liquid with resistivity Re1, and the second reservoir has a test material or test liquid with resistivity Re2, and that the main electrode (11) makes electrical contact with the test material or test liquid with negligible contact resistance and negligible electrode resistance, the test resistance of the lumen part (RL) corresponds to θ/2 times the test resistance of the aperture part (RA), where θ=Re2/Re1, and θ*R_{A}/R_{L} ranges from 0.27 to 23. Here, Re1 represents the resistivity of the first reservoir, and Re2 represents the resistivity of the second reservoir.

In embodiments, the lumen part may have a resistance R_{L} and the aperture part has a resistance R_{A}, or wherein the pore part located above the electrical center, on the lumen side, has a resistance R_{c} and the pore part located below the electrical center, on the aperture side, has a resistance Rₚ, where the geometry independent of electrolyte conforms to:

$$\frac{F / 4}{rtop} + F {\int }_{electrode top}^{top} \frac{1}{A ( l )} dl = {\int }_{bottom}^{electrode bottom} \frac{1}{A ( l )} dl + \frac{1 / 4}{rbottom}$$

wherein A(l) is the area of the cross section at position l along the pore, and top, bottom, electrode bottom and electrode top correspond to positions along the pore, where electrode refers to the main electrode, where rtop and rbottom is the effective cross-section radius at the ends of the pore, and wherein F is from 0.27 to 23, for example 0.57 to 8.6. Rₚ/R_{c} is from 0.27 to 23.

In embodiments, the lumen part of the pore may comprise two sub-parts: a first sub-lumen connected to the first opening, and a second sub-lumen connected to a third opening. The device may further include a third reservoir, fluidically connected to the second sub-lumen of the pore via the third opening. The pore is fluidically connected to the first, second, and third reservoirs.

In embodiments, the main electrode may be located on the walls of the pore, with at least ¾ of the electrode area exposed to the lumen part of the pore.

In embodiments, a thin solid dielectric material may be present between the electrode and the space inside the pore. The thin solid dielectric material may have a thickness of 0.5-10 nm and may be made of materials such as Al₂O₃, TiO₂, ZrO₂, HfO₂, Si₃N₄, or SiO₂.

In embodiments, the main electrode may be made of an electrically conducting material, such as TiN, Ru, Pt, or silicon.

In embodiments, the main electrode may be a semiconductor of a metal-oxide-semiconductor field-effect transistor (MOSFET) channel, with a dielectric covering the electrode serving as a gate dielectric.

In embodiments, the main electrode may be electrically connected to a gate of a MOSFET.

In embodiments, the aperture may have a height (h_{A}) and an effective diameter (d_{A}), with h_{A} being less than 50 nm and dA being less than 20 nm.

In embodiments, the lumen may have an effective diameter (d_{L}), ranging from 70 to 1000 nm.

In embodiments, the device may include a dielectric wall of thickness (w) surrounding the pore, where the total summed capacitance between the electrolyte inside the pore and the outside electrical conductors (including the first and second reservoirs and the main electrode) is smaller than 50 fF.

In embodiments, the device may further comprise a semiconductor wall of thickness W_{Si} surrounding the pore, wherein the wall thickness W_{Si} is 10 nm or less, such as 5 nm or less.

In embodiments, the wall may comprise at least three different layers consisting of at least two different materials. These layers are arranged on top of each other, with a dielectric material at the top and bottom of the pore. The top dielectric layer has a height (h_{ox_top}), the bottom dielectric layer has a height (h_{ox_bottom}), and a conductive/semiconductive material is arranged in the middle of the pore, with a height (hₛᵢ).

In embodiments, the lumen may have a height (h_{L}) equal to h_{ox_top}+hₛᵢ, and the aperture may have a height (h_{A}) equal to h_{ox_bottom}.

In embodiments, the conductive/semiconductive material may have a height (hₛᵢ) ranging from 5 to 500 nm, such as 5 to 100 nm.

In embodiments, the lumen of the pore may have a uniform shape or a tapered shape.

In embodiments, the device may include a main electrode and one or more additional electrodes, which may be located in the aperture part or the lumen part of the pore.

In embodiments, the pore may be embedded in the FET, with the FET being gated by an electrolyte-filled pore running through the channel region of the FET. The sensor region may comprise a source-drain axis.

In embodiments, the semiconductive material may be silicon (Si).

In embodiments, a fourth layer may be arranged below the bottom dielectric material, with a height (h₄). In this case, h_{L}=h_{ox_top}+hₛᵢ+h_{ox_bottom}, and h_{A}=h₄.

In embodiments, the fourth layer may be a dielectric multi-layer consisting of multiple different dielectric materials.

In embodiments, the fourth layer may be a 2D material.

In embodiments, the device may include a remote extended FET, where an electrode wrapped around the nanopore is connected to a remote gate sensor.

In embodiments, the wall material and conductive material may be an electrode material, such as titanium nitride (TiN), ruthenium (Ru), or platinum (Pt), which is coupled to a gate of a silicon transistor remote from the pore.

In some aspects, the present disclosure may relate to a system for sensing target molecules in an electrolyte, the system including a first reservoir comprising target molecules in an electrolyte, a second reservoir, and optionally additional reservoirs. The system may also include a pore FET device as described in the previous embodiments, with the pore connecting the first and second reservoirs. The sensing of target molecules involves the target molecules exiting the first reservoir, entering the lumen part of the pore via the first opening, passing through the pore along the pore axis, and exiting through the second opening of the aperture part into the second reservoir (or vice versa). The electrolyte in the lumen has a resistance R_{L}, while the electrolyte in the aperture has a resistance R_{A}. A significant modulation of the resistance R_{A} occurs when a target molecule translocates through the aperture, while no significant modulation of the resistance R_{L} occurs when a target molecule translocates through the lumen. For instance, the system may be for sensing of target molecules in an electrolyte, the system comprising: i) a first reservoir comprising target molecules in an electrolyte; ii) a second reservoir; iii) optionally additional reservoirs; iv) a nanoscale pore Field-effect transistor (FET) device as defined above and below, comprising a nanopore connecting the first and second reservoir, wherein the sensing of target molecules comprises the target molecules exiting the first reservoir, entering the lumen part of the nanoscale pore of the FET device via the first opening, passing through the nanoscale pore along the pore axis and exiting through the second opening of the aperture part into the second reservoir or vice versa, and wherein the electrolyte in the lumen has a resistance R_{L} and the electrolyte in the aperture has a resistance R_{A}, such that a significant modulation of the resistance R_{A} occurs when a target molecule translocates through the aperture, while no significant modulation of the resistance R_{L} occurs when a target molecule translocates through the lumen.

In embodiments, the resistance of the parts of the pore, R_{A}, R_{L}, R_{P}, and R_{C}, may be tuned by adapting the geometry of the device, the concentration of a carrier in the electrolyte, and the surface charge.

In other aspects, is further provided a simulation method for designing a pore FET device that involves providing a pore FET device as described in the previous embodiments, uniformly filling the pore and the first and second reservoirs with a single isotropic and uniformly conducting test material or test liquid, arranging the main electrode to make electrical contact with the test material or test liquid with negligible contact resistance by removing any thin dielectric on the main electrode, and adapting the geometry of the device such that either the resistance between the second reservoir and the main electrode (R1) corresponds to the resistance between the first reservoir and the main electrode (R2), with R_{A}/R_{L} ranging from 0.27 to 23, or the test resistance of the lumen part (R_{L}) corresponds to θ/2 times the test resistance of the aperture part (R_{A}), where θ=Re2/Re1, and θ*R_{A}/R_{L} ranges from 0.27 to 23. The first reservoir has a resistivity Re1, and the second reservoir has a resistivity Re2. For instance, the simulation method may be for designing a nanoscale pore Field-effect transistor (FET) device, the method comprising: providing a nanoscale pore (FET) device as defined above and below; uniformly filling the nanoscale pore and the first and second reservoirs with a single isotropic and uniformly conducting test material or test liquid; arranging the main electrode to make electrical contact with the test material or test liquid with negligible contact resistance by removing any thin dielectric on the main electrode, and adapting (S3) the geometry of the device such that: i) the resistance between the second reservoir and the main electrode, R1 corresponds to the resistance between the first reservoir and the main electrode, R2, such that R1 and R2 are not different by more than a factor of 6 or less than a factor of ⅙; or ii) the test resistance of the lumen part, R_{L}, corresponds to θ times the test resistance of the aperture part, R_{A}, wherein

$$θ = \frac{Re 2}{Re 1} ,$$

such that θ*R_{A} and R_{L} are not different by more than a factor of 6 or less than a factor of ⅙ (do not vary more than within a factor of ⅙ to 6), wherein the first reservoir has a resistivity Re1, and the second reservoir has a resistivity Re2. The test resistance is the resistance that results from the simulation or experiment. Adapting in this case means adapting the geometry (size) of the parts of the device, especially the lumen part and aperture part.

### BRIEF DESCRIPTION OF THE DRAWINGS

The above, as well as additional objects, features, and advantages of the present inventive concept, will be better understood through the following illustrative and non-limiting detailed description of different embodiments of the present inventive concept, with reference to the appended drawings, wherein:

FIG. 1 illustrates a schematic overview of the pore sensor, the pore having a lumen part and an aperture part separated by an electrical center, where the resistance above the electrical center (of the lumen part) is approximately half of the resistance below the electrical center (of the aperture part). Furthermore, cross-sections across the width of the pore are illustrated, being oriented substantially orthogonally to the length of the pore, having a pore axis running along the length of the pore, according to an example embodiment.

FIG. 2 illustrates a schematic cross-section of a pore, according to an example embodiment.

FIG. 3A illustrates a schematic cross-section of a pore where the aperture and lumen part are vertically oriented, while FIG. 3B shows a lateral design where the lumen is rotated 90° so that it is perpendicular to the aperture, according to an example embodiment.

FIG. 4 illustrates an example of a lateral device design, according to an example embodiment.

FIG. 5 illustrates a further example of a lateral design, according to an example embodiment.

FIG. 6 illustrates a further example of a lateral design comprising multiple sub-lumens, according to an example embodiment.

FIG. 7 illustrates the correlation between the resistances Rc/Rp and the signal modulation, according to an example embodiment.

FIG. 8 illustrates schematic cross-sections of the asymmetric device design for a silicon pore FET (embedded nanopore), where FIG. 8 (left) shows a side view (longitudinal cross-section) of the device, while FIG. 8 (right) shows atop view (horizontal cross section) of the device, according to an example embodiment.

FIG. 9 illustrates an alternative embodiment of the device in FIG. 8, where a fourth layer may be arranged below the bottom dielectric material. FIG. 9 (left) shows the introduction of a dielectric multi-layer consisting of multiple different dielectric materials, while 9 (right) shows the introduction of a 2D material below the bottom oxide, according to an example embodiment.

FIG. 10 illustrates schematic cross-sections of the asymmetric device design for an extended gate pore FET, where 10 (left) shows a side view (longitudinal cross-section) of the device, while 10 (right) shows a top view (horizontal cross section) of the device, according to an example embodiment.

FIGS. 11A-D illustrate alternative designs of the present devices, where the location of the main electrode or conductive layer acting as channel may be varied, according to an example embodiment.

FIGS. 12A-B illustrate alternative designs of the present devices, where several conductive layers acting as channel or electrode connected thereto may be used, and that the location of these may vary, according to an example embodiment.

FIG. 13 illustrates the pore bandwidth (BW) and SNR trade-off. FIG. 13 (left) shows the impact of the design measures on the SNR, while FIG. 13 (right) illustrates the impact of the design measures on the BW, according to an example embodiment.

FIG. 14 shows the bandwidth at SNR=1 of an extended gate FET versus electrode area and FET area, according to an example embodiment.

FIG. 15 shows the lumen length versus lumen diameter for different aperture sizes, according to an example embodiment.

The figures are not necessarily to scale, and generally only show parts that are necessary in order to elucidate the inventive concept, wherein other parts may be omitted or merely suggested.

### DETAILED DESCRIPTION

Aspects of the present disclosure will be described more fully hereinafter with reference to the accompanying drawings. The devices and systems disclosed herein can, however, be realized in many different forms and should not be construed as being limited to the aspects set forth herein. Like numbers in the drawings refer to like elements throughout.

The terminology used herein is for the purpose of describing particular aspects of the disclosure only and is not intended to limit the disclosure. As used herein, the singular forms “a”, “an” and “the” are intended to include the plural forms as well, unless the context clearly indicates otherwise.

In some embodiments, a non-limiting term “molecule” as in “target molecule” is used. This refers to any type of analyte that may be detected, identified, and quantified using the device and system of the present disclosure.

In some embodiments a non-limiting term “biomolecule” is used. The term “biopolymer” may also be used interchangeably. The term “target biomolecule” or “target biopolymer” may be used, and thus refers to the biomolecule/biopolymer that the present embodiments tries to sense/detect. The target biopolymers or biomolecules herein can be any type of biopolymers, such as polynucleotides, polypeptides, lipids, or polysaccharides, including DNA- or RNA-polymers, peptides, and proteins.

A semiconductor material, such as silicon, has an electrical conductivity value falling between that of a conductor and an insulator, and may conduct electric currents in the solid state. A transistor is a semiconductor device used to amplify or switch electrical signals and power. A field-effect transistor (FET) is a type of transistor that uses an electric field to control the flow of current in a semiconductor. FETs, such as junction-gate field-effect transistors (JFETs) or metal-oxide-semiconductor field-effect transistor (MOSFETs) are devices with three terminals: source, gate, and drain. FETs control the flow of current by the application of a voltage to the gate, which in turn alters the conductivity between the drain and source. MOSFET is a type of field-effect transistor which is most commonly fabricated by the controlled oxidation of silicon. It has an insulated gate, the voltage of which determines the conductivity of the device. This ability to change conductivity with the amount of applied voltage can be used for amplifying or switching electronic signals. Although using the term “metal” in the name MOSFET, the gate material can be a layer of polysilicon (polycrystalline silicon). Similarly, regarding “oxide”, different dielectric materials are used with the aim of obtaining strong channels with smaller applied voltages. Typically, Silicon oxide (SiOₓ), such as Silicon dioxide (SiO₂) may be used as an insulation layer between the gate and the conducting channel of the MOSFET. A reason to use SiO₂ is that it provides good isolation and that SiO₂ is a good dielectric material, where it is only needed to perform oxidation on an already grown Si layer to achieve isolation.

The terms cross-section and transverse cross-section, when referring to the pore, are used interchangeably and refer to the shape of the pore when cut perpendicular to its axis or central pathway.

The term ‘pore’ is used throughout this document to describe a structure characterized by an effective transverse cross-sectional diameter that is consistently less than 1 μm along its entire length. Additionally, the pore possesses at least one region where the effective transverse cross-sectional diameter is less than 50 nm. Given these dimensions, the pore qualifies as both a nanopore and a nanoscale pore.

As used herein, the term “pore field-effect transistor” should be construed as a FET, wherein a pore is used for providing a gate voltage to the FET. The pore could be separately arranged from the channel region of the FET. For instance, the pore could be provided with an electrode which is directly connected to a gate electrode of the FET arranged close to the channel region, such that a voltage of the gate electrode could be controlled by the electrode of the pore. However, according to an alternative, the pore may be arranged in relation to the channel region, such that the pore may control the gate voltage directly, whereby the gate material is represented by the fluid inside the pore. For instance, the pore may be configured to extend through the channel region.

The features “nanopore-FET” (NP-FET), “nanopore-FET device”, “nanoscale pore-FET” or “nanoscale pore-FET device” may be used interchangeably herein and refers to a device comprising a nanopore/nanoscale pore and a FET sensor, where the FET may be either embedded in the nanopore, or remote, i.e. where an electrode wrapped around the nanopore is connected to a remote gate sensor. Thus, in one embodiment, nanoscale FETs gated by an electrolyte-filled nanopore running through the channel of the FET is provided, typically a silicon gate. Alternatively, an electrode is wrapped around the nanopore, which is connected to a remote FET, thus having an extended gate.

Nanopores are pores of nanometer size that may be created by a pore-forming protein or as a hole in synthetic materials, such as silicon, silicon nitride or graphene. Typically, nanopores are quite narrow, with larger nanopores being up to 20 nm in effective diameter. However, in the current disclosure, the pores may be up to 1 micron in effective diameter, and thus, to emphasize the possible size of the pores, the term “nanoscale pore” is used interchangeably to nanopore herein. Nanopores may be organic when formed by pore-forming proteins, or they may be inorganic solid-state nanopores made for example in silicon compound membranes. The lack of large-scale tunability of the diameter of biological nanopore provided a motivation for the development of synthetic nanopores. Synthetic nanopores can be fabricated in virtually any size from below 1 nm in effective diameter to the sub-micrometer range. These sizes allow for the analysis of a large range of biomolecules including proteins, viruses, and nanoparticles. Nanoscale pores or channels in solid state materials are fabricated by various means including ion beam sculpting, focused ion beam fabrication, transmission electron microscopy, electron beam fabrication, track-etching, dielectric break down, laser-assisted dielectric breakdown, laser-assisted etching, and layer-by-layer removal. These techniques make it possible to create nanopores with varying shapes and surface chemistries in a range of materials including silicon nitride, silicon dioxide, hafnium oxide, aluminum oxide, graphene, glass and polymer films. Nanopores have been shown to function as a single-molecule detector when present in an electrically insulating membrane, where a voltage is applied across the membrane and an ionic current passing through the nanopore is monitored. The membrane, biological or solid-state, comprising the nanopore is surrounded by an electrolyte solution, where the membrane may split the solution into two chambers. An electric field is attained by applying a bias voltage across the membrane, inducing an electric field that drives motion of charged particles (ions). In some example embodiments, all the voltage drop concentrates near and inside the nanopore such that charged particles in the solution only feel a force from the electric field when they are near the pore region, also denoted capture region. Nano-sized polymers, such as DNA or protein having a net charge, will then, if placed in one of the chambers, feel a force from the electric field when near the pore region. Thus, the molecule will be attracted to the pore capture region, enter the nanopore, and inside the nanopore translocate through via a combination of electro-phoretic, electro-osmotic and sometimes thermo-phoretic forces. The nanopores/nanoscale pores of the present disclosure are sometimes referred to as “pore”, and the pore may also refer to the inside of the nanopore, the nanopore channel, which has a pore axis running along the pore. Typically, in the present devices, the target molecules enter the device at the lumen part, but optionally the molecules may enter the pore at any opening. The nanoscale pore of the present devices may be a hybrid pore in a solid-state pore, such as comprising a biopore.

For a nanopore of molecular dimensions, passage of molecules, such as DNA, cause interruptions of the current level of the open pore, leading to a signal as the molecule translocates through the pore. Inside the pore the molecule occupies a volume that partially restricts the flow of ions, observed as an ionic current drop. The characteristics of the said interruption or disruption of the current may then be used to determine the sequence or identity of the molecule passing through the pore. Based on various factors such as geometry, size and chemical composition, the change in magnitude of the ionic current and the duration of the translocation will vary. Different molecules can then be sensed and potentially be identified based on this modulation in ionic current, which is referred to as nanopore sensing.

It has previously been suggested to combine nanopore sensing with field effect transistor (FET) devices. Methods of using a sensor comprising a FET embedded in a nanopore may for example include placing the sensor in an electrolyte comprising at least one of biomolecules and deoxyribonucleic acid (DNA), placing an electrode in the electrolyte, applying a gate voltage in the sub-threshold regime to the electrode, applying a drain voltage to a drain of the FET, applying a source voltage to a source of the FET, detecting a change in a drain current in the sensor in response to the at least one of biomolecules and DNA passing through the nanopore. In some embodiments, the nanopore FET is a transistor wrapped around a nanopore connecting two electrolyte reservoirs. The nanopore FET can detect target molecules passing through the nanopore as a change in a source-drain current of the nanopore FET. Designing dimensions of the nanopore FET involves selecting geometry for formation of a carrier channel in semiconductor around the pore while avoiding leakage current from source to drain. Improved sensitivity can be expected when potential perturbations by molecules in the nanopore impact electron density in the channel maximally. Sensitivity of the nanopore FET can be defined as maximal change in current due to presence of the molecule normalized by magnitude of the current. Thus, in some aspects are provided a pore field-effect transistor sensor for sensing target molecules, wherein the pore field-effect transistor sensor comprises a field-effect transistor having a source, a drain, a dielectric, a gate, and a channel region between the source and the drain, wherein the gate is controlled by a pore filled by an electrically conducting fluid (electrolyte) and the pore is configured to extend from a first side to a second side to provide gating of the field-effect transistor from within the pore, and wherein the pore field-effect transistor sensor further comprises a first electrode for setting a first voltage at the first side of the pore and a second electrode for setting a second voltage at the second side of the pore, wherein the molecule to be sensed passing through the fluid in the pore is configured to modulate the drain-source current for detecting presence of the molecule.

The pore is configured to allow the target molecule to be detected/sensed to pass through the pore. Further, the pore is configured to have a cross-sectional dimension (effective diameter/area) at least in one location of the pore, such that the molecule to be detected blocks the pore to such an extent that potential perturbations within the pore are generated and the effective gate voltage of the FET is changed. Thus, when the molecule to be detected passes through the pore, the effective gate voltage of the FET will be changed such that the drain-source current of the FET will be modulated. This enables detecting the molecule being passed through the pore.

The pore may be filled by a fluid during operation such that the molecule to be detected may be transported through the fluid for passing through the pore. A sample to be analyzed, potentially comprising the target molecule, may be inserted into the fluid which is previously provided in the pore FET sensor. Alternatively, the sample may be introduced with the fluid into the pore FET sensor. The fluid (and the sample) may further be removed from the pore FET sensor and replaced so as to allow the pore FET sensor to analyze further samples. The FET may be a metal-oxide-semiconductor field-effect transistor (MOSFET) to provide current through the transistor in the subthreshold region. According to an embodiment, a voltage difference between the first voltage and the second voltage additionally drives the particle to be detected through the pore. It should be realized that the voltage difference between the first voltage and the second voltage may drive the molecules to be detected through the pore. If they are positively charged, they may be driven from the first side to the second side of the pore towards the lower potential set by the second voltage. If the molecules are negatively charged, they may be driven from the second side to the first side. Also, the electrically conducting fluid may be provided with salt ions (or other ions) such that presence of an electric field in the fluid due to the difference between the first voltage and the second voltage may create a force on the fluid to generate a movement in the fluid that may drag along uncharged parts of the fluid, such as uncharged particles to be detected. This implies that the voltage difference provided for enabling detecting the particle to be detected with a high sensitivity may also control flow of the particle to be detected through the pore. However, it should be realized that alternatively or additionally, the driving of particles to be detected through the pore may be provided by in other manners. This may be particularly useful for driving uncharged particles to be detected. For instance, driving of particles may be provided by a pressure difference between the first side and the second side of the pore and/or by a difference in concentration of the particles to be detected or of ions in the fluid, resulting in a fluid flow through the pore dragging particles to be detected along the fluid flow.

Thus, there has been increasing interest in bringing together both field effect transistors (FETs) and nanopores to develop pore FETs to sense target molecules, such as embedding a FET in the nanopore. The physical principles of electrolytically gated pore FETs are similar to that of the more conventional semiconductor FETs with the exception that the gate consists of an electrolyte containing ions rather than a solid-state metal or highly doped poly-silicon gate. Such platforms could enable improved selectivity and controlled molecular transport; however, challenges remain including fabrication, operational stability, and ease of functionalization.

One implementation of a nanopore FET (NPFET) is a device with a nanopore embedded within the channel of the FET. This is a promising new device detecting the motion of a molecule through the nanopore based on the transistor's electronic current. This nano-scale FET-based approach may enable next-generation single-molecule sensing by offering larger signals and hence higher bandwidth, responding to a key challenge of detecting fast translocating molecules, and by offering denser electronic system packing and therefore more parallel sensing.

However, especially when using a larger bandwidth for these devices, a problem with noise occurs. To obtain a sufficiently high bandwidth, the active area of the FET on the inside of the nanopore should be kept as small as possible, and for picking up an as large as possible signal due to translocating molecules the nanopore should be as small as possible, on the nanoscale, while for keeping the noise down, the size and area of the FET inside the nanopore, more particularly of the sensitive surface of the FET inside the nanopore should be large. Thus, it is challenging to obtain FET-based nanopore sensors with sufficient SNR and short channel control, or good coupling to a remote FET, which all demand a larger nanopore, as well as high sensitivity to short molecules, high resolution and high bandwidth, which all demand smaller nanopores. Thus, the current disclosure relates to solving the problem of these opposite demands on the device. Here we provide a nanopore solution which satisfies these seemingly opposing sets of demands.

Accordingly, the present disclosure demonstrates how to combine a nanopore (a nanoscale pore) with FET-based sensing to read out molecules translocating through the nanopore at higher bandwidth than is possible when reading out ionic currents. Herein are thus provided new nanopore FET devices, which are nanoscale FETs gated by an electrolyte-filled nanopore running through the channel of the FET, or alternatively, an electrode wrapped around the nanopore may be connected to a remote FET. The key problem solved by these new devices is how to maintain low noise sensor characteristics while still obtaining a very strong translocation signal magnitude and while also discriminating or resolving sub-molecular features spaced apart on a nanometer scale on a (bio)polymer translocating the pore, and while also maintaining a bandwidth improvement (>1-10 MHz bandwidth with SNR>1).

The device of the present disclosure demonstrates how to combine a nanopore with FET-based sensing to read out molecules translocating through the nanopore at higher bandwidth than is possible when reading out ionic currents, while maintaining low noise characteristics. One implementation of a pore (e.g., nanopore) FET is a FET (e.g., nanoscale FET) gated by an electrolyte-filled pore running through the channel of the FET. The pore FET (e.g., nanopore FET) may comprise a sensor region, having a source region and a drain region, defining a source-drain axis, with a channel region between the source region and the drain region, where the pore (e.g., nanopore) may be defined as a an opening in the channel region which completely crosses through the channel region, oriented at an angle (such as orthogonally) to the source-drain axis, the nanopore may have multiple openings, such as having a first opening and a second opening, where the first opening is an opening between a lumen part of the pore (e.g., nanopore) and a first reservoir, which may be referred to as a lumen reservoir, and the second opening between an aperture part of the nanopore and a second reservoir, also referred to as an aperture reservoir. Alternatively, an electrode wrapped around the pore (e.g., nanopore) may be connected to a remote FET.

The key problem solved by the devices of the present disclosure is how to maintain low noise sensor characteristics while still obtaining a very strong translocation signal magnitude and while also discriminating or resolving sub-molecular features spaced apart on a nanometer scale on a (bio)polymer translocating the pore, and while also maintaining a bandwidth improvement (>1-10 MHz bandwidth with SNR>1). For a nanopore FET with a nanopore running through the nanoscale FET's channel or for a nanopore running through a nanoscale electrode connected to a nanoscale FET, a sufficiently high bandwidth can be obtained if the active area of the FET or electrode, on the inside of the nanopore, is kept sufficiently small, such as on a sub-micron scale. To obtain a molecular signal with high magnitude the nanopore may be as small as possible, on the nanoscale (<50 nm), both in terms of effective diameter and height. A low height may allow for high resolution, where resolution is the ability to resolve closely spaced features on a molecule. When the nanopore runs through the FET, the nanopore also determines the effective channel length and the area of the FET, as here the FET's surface is formed by the inside of the nanopore. Hence the effective length and area are desired to be as small as possible for the signal and bandwidth. This poses a problem. The size and area of the FET, or of an electrode connected to the gate of a remote FET, is important for the noise of the sensor, the larger the area, the less noise, and low noise is critical for sensors. Moreover, nanoscale FETs suffer from short channel effects unless a particular geometric design is used, and these short channel effects will grow worse for smaller channel lengths. The nanopore sizes desired for large signal and high-resolution result in poor FET characteristics due to short channel effects. Short-channel effects occur when the channel length is the same order of magnitude as the depletion-layer widths of the source and drain junction. In MOSFETs, channel lengths must be greater than the sum of the drain and source depletion widths to avoid edge effects. Otherwise, a number of effects may appear, such as “off-state” leakage current, impact ionization, in which a charge carrier can be affected by other charge carriers, velocity saturation/mobility degradation, drain-induced barrier lowering (DIBL), which is caused by encroachment of the drain depletion region into the channel, drain punch through, whereby current flows regardless of gate voltage—a phenomenon that can occur if the drain is at high enough voltage compared to the source and the depletion region around the drain extends to the source, surface scattering, channel length modulation and threshold voltage roll-off. Other problems arise due to the small size of the FET as well, such as increased variability.

For remote gate sensors, the coupling to a remote FET will improve for larger electrode areas in the nanopore as well. Better coupling via a capacitive divider will improve sensitivity. The key challenge is how to obtain low noise (and suppress short channel effects or improve coupling) making use of such an unconventional nanopore gate, while also still obtaining a strong signal and resolution of molecules translocating through the nanopore, as well as while maintaining a bandwidth improvement.

Accordingly, new nanopore/pore FET devices, having an embedded FET or a remote FET, have been designed, the new FET-based pore (e.g., nanoscale pore) sensor devices having improved noise characteristics (as well as suppressed short channel effects for silicon gate, and better coupling for extended gate), high translocation signal magnitude and high bandwidth. These properties are achieved by adjusting the properties, including geometry, of the device, as described below.

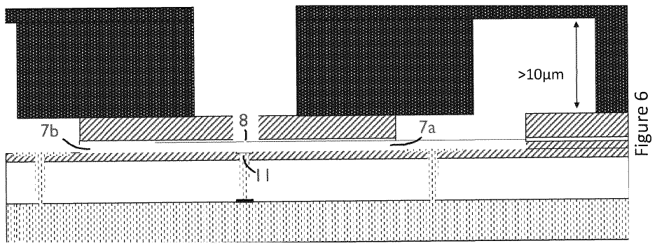

The pore (e.g., nanoscale pore) of the FET-based pore (e.g., nanoscale pore) device comprises two main contiguous parts, a lumen part and an aperture part or aperture, wherein each part contains one end (or opening) of the pore. One end of the aperture pore part is fluidically connected to an aperture reservoir. The pore (e.g., nanoscale pore) further comprises a plurality of openings. In an embodiment, the pore (e.g., nanoscale pore) comprises two openings, a first and a second opening, such that the lumen part is connected to the first opening and the aperture part is connected to the second opening. In other embodiments, the lumen part comprises several sub-lumen parts, each in connection (e.g., direct fluidic connection) with an opening of the pore (e.g., nanoscale pore). Thus, the lumen part may comprise multiple sub-lumens each fluidically connecting the other end of the aperture part (the end not connected to the aperture reservoir) to a (different) reservoir (excluding the aperture reservoir). In an embodiment, the lumen part comprises two sub-parts, a first sub-lumen and a second sub-lumen, wherein the first sub-lumen is connected (e.g., directly fluidically connected) to the first opening and the second sub-lumen is connected to a third opening. The openings in the pore (e.g., nanoscale pore) leads to a plurality of reservoirs, such as one reservoir per opening, wherein the inside of the pore (e.g., nanoscale pore) is fluidically connected to the reservoirs. With multiple reservoirs, one reservoir is connected to the aperture parts, and the other to multiple sub-lumen parts. In one embodiment, the device comprises a first and a second reservoir, the pore (e.g., nanoscale pore) being fluidically connected to the first and second reservoir, wherein the first reservoir is in connection with the lumen part of the pore (e.g., nanoscale pore) via the first opening, and the second reservoir is in connection with the aperture part of the pore (e.g., nanoscale pore) via the second opening. In another embodiment the device comprises a third reservoir and the lumen part comprises two sub-parts, a first sub-lumen and a second sub-lumen connected to the first and third openings, respectively, wherein the third reservoir is in connection with the second sub-lumen of the pore (e.g., nanoscale pore) via the third opening, and wherein the pore is fluidically connected to the first, second and third reservoir. Such design with two sublumens may allow for improvements in terms of wetting and fluidics since liquid can flow through the lumen from reservoir 1 to reservoir 3 rendering the wetting of the lumen easier compared to a single-ended lumen by avoiding the formation of air bubbles when wetting the lumen. A liquid flow can be induced through the lumen by applying a hydrostatic pressure difference across the lumen between reservoir 1 and 3. Moreover, such flow allows to supply and evacuate analytes more easily. An example of such a flow-through lumen design is illustrated in FIG. 6.

The device further comprises a pore axis oriented from the first opening to the second opening of the pore (e.g., nanoscale pore), wherein the one or more lumen parts and the aperture part of the pore (e.g., nanoscale pore) are oriented along the pore axis. In other words, the device further comprises a pore central pathway running from the first opening to the other openings of the pore, wherein the one or more lumen part and the aperture part of the pore are oriented along the pore central pathway. It should be noted that the pore axis is not necessarily linear, but may be bent, and also it may be split if the pore comprises more than two openings, such that the pore axis may head in two different directions towards different openings. Thus, the pore axis may be seen as a curve running through the center of the pore tubular structure. The center is the center of mass of the cross-section, or 2D cross-sectional shape (assuming uniform mass density) in a plane orthogonal to the pore axis. Target molecules may translocate through the nanopore along the pore axis, e.g. from a first reservoir into a second (or further) reservoir. In some embodiments, the target molecules enter the pore (e.g., nanoscale pore) from a first reservoir via a first opening in the lumen part and exit the nanopore via the second opening in the aperture part, where the lumen part and aperture are oriented along the pore axis. In other words, the device includes a pore central pathway that extends from the first opening to the second opening of the pore. This pathway serves as a reference line for the orientation of the one or more lumen parts and the aperture part of the pore. The central pathway through the pore need not be straight and may even divide into branches. In embodiments, the central pathway may comprise a straight axis or a plurality of straight axis connected to one another at an angle or via a curved section. When the pore has more than two openings, multiple straight axes may be defined, each extending toward different openings. These axes can be understood as straight lines that pass through the center of mass of the pore's cross-sectional shape, assuming uniform mass density, in a plane that is perpendicular to each straight axis. Target molecules may move along pathways that are generally aligned with these axes, for example, transitioning from a first reservoir to a second or subsequent reservoir.

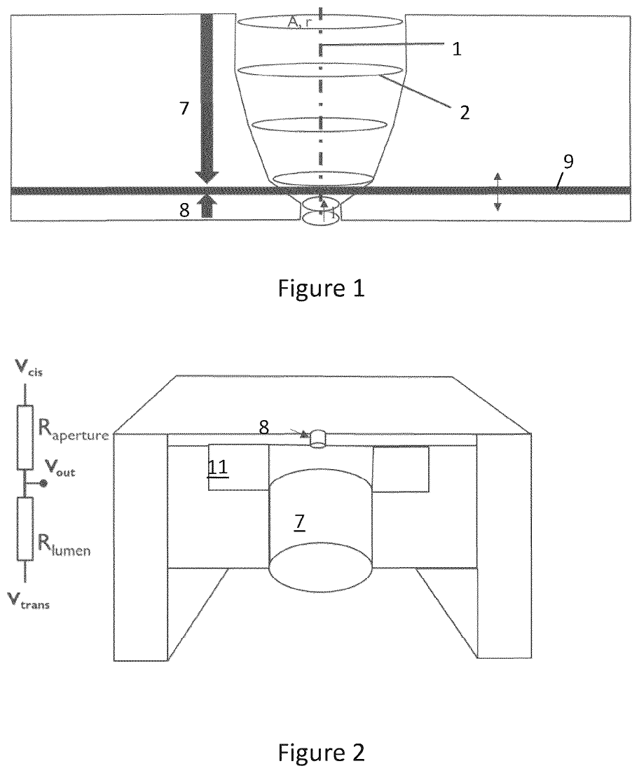

The device may further comprise an electrical center (9, see FIG. 1) and at least one sensor region. The at least one sensor region is such that a target molecule travelling along the pore axis (i.e., passing through the pore) will pass through the sensor region (sensitive region). The sensor region may also be referred to as an electrode region. The sensor region of the pore comprises a sensitive gate surface exposed to the electrolyte of the pore. When the nanopore is embedded in the FET, such that the FET is directly gated by an electrolyte-filled nanopore running through or beside the channel region of the FET, the sensor region is in between a source and drain region (source drain axis). The sensor region then comprises the semiconductor channel of the FET with the nanopore running through or beside the semiconductor channel. The channel has an electrical contact to both the source region and the drain region. When the FET is instead remote, there are no source and drain regions near the sensor region, but the sensor region comprises a conducting (e.g. metal) electrode with the nanopore running through or beside it, and one contact to the electrode can suffice. This contact is in electrical connection to the gate of a remote FET.

When the pore is embedded, the electrical center and the sensor region overlaps. Thus, in this embodiment, the device comprises an electrical center comprising a sensor region of the device, the sensor region comprising the main electrode located between, and potentially overlapping, the lumen part and the aperture part of the pore (e.g., nanoscale pore). In some example embodiments, the main electrode overlaps at least one of the lumen parts and the aperture part of the pore. This sensor region is mostly in the lumen region to allow for a sufficiently large electrode size to enable low noise. The main electrode area is exposed for at least three quarters to the lumen part of the pore. The location of the sensor region mostly in the large lumen region importantly allows for a larger sensitive gate surface area then would be possible in the aperture region. The larger sensitive gate surface area allows low noise while still being sufficiently small to attain high bandwidths. The main electrode is made of an electrically conducting or semi-conducting material, such as but not limited to TiN, Ru, Pt, and silicon. A thin solid dielectric material (0.5-10 nm), such as but not limited to Al₂O₃, TiO₂, ZrO₂, HfO₂, Si₃N₄, SiO₂, may be present between the electrode and the space inside the pore. The main electrode is typically located on the wall of the pore. The main electrode is either the semiconductor of a metal-oxide-semiconductor field effect transistor (MOSFET) channel whereby the dielectric covering the electrode is the gate dielectric or it is electrically connected to the gate of a metal-oxide-semiconductor field effect transistor (MOSFET). In this last case, the electrode is electrically conducting. Additional electrodes may be present apart from the main electrode. Thus, in some embodiments the device comprises a main electrode and one or more additional electrodes. The additional electrode may be located in the aperture part or the lumen part of the nanopore.

The sensor region design of electrical center in the lumen has been “decoupled” from the aperture. The aperture determines the translocation signal. The decoupling enables an aperture with a small effective diameter and low height (<50 nm) for a strong molecular signal, and a lumen, containing the sensor region, with larger effective diameter and height for low noise. The maintenance of a sufficiently small scale (<1 um) for the lumen allows for maintaining high bandwidth and high integration density.

The pore axis runs parallel to ion flow direction, through the center of cross sections of the pore, where the cross-sections are defined as a 2D shape formed by a cross-section of the pore tubular structure and a plane orthogonal to the nanopore axis running through the point on the pore axis. It should be appreciated that the pore axis does not necessarily need to be linear, but can also be bent, as discussed above. The lumen part and aperture part meet near the electrical center, which is the main electrode position, such that the aperture part is situated mainly “below” the main electrode, such that less than 25% of the electrode area of the electrical center is in the aperture part, and the lumen part is situated mainly “above” the main electrode, where the electrode may be situated entirely in the lumen part, and there may be a spacer in the lumen part separating the electrode from the aperture part.

By adjusting the size of the lumen part and the aperture part of the pore (e.g., nanoscale pore) in the device, such that the lumen is long and wide, and the aperture is short and narrow, both high bandwidth and low noise may be attained. The asymmetric design allows to decouple the FET design in the lumen from the aperture, where the aperture determines the translocation signal. In this way the FET can be made larger to control the short channel effects, while still maintaining a sufficient translocation signal, because the aperture can be made very small without affecting FET properties. The FET active area determines the bandwidth but can still be kept sufficiently small to realize bandwidths >1-10 MHz for which SNR>1. Hence the devices of the present disclosure allow a high-quality FET with suppressed short channel effects, a sufficient signal magnitude and high resolution nanopore detector and high bandwidths (>1-10 MHz bandwidth with SNR>1).

An embodiment of the present disclosure is illustrated in FIG. 1, showing the lumen part (7) of the nanopore as the wide and long part of the pore, while the aperture part (8) is narrow and short. A cross-section may be made at a point on the pore axis; a 2D shape formed by the cross-section of the pore tubular structure and a plane orthogonal to the nanopore axis (i.e., the pore central pathway) (1) running through the point on the pore axis. An effective cross-sectional radius may thus be defined as r=\sqrt{A/π}, with A the cross-sectional area, and an effective cross-sectional diameter d=2r. The size of the different parts of the nanopore is such that cross sections orthogonal to the flow direction along the pore axis (the direction of the target molecules translocating through the pore) have an area, A, defined by

A=πr², [Equation 1]

r being the effective cross-sectional radius of each cross section, the cross sections approximately having an equipotential surface. It should be appreciated that the inside of the pore may not be fully circular, but may for instance be square or have other shapes. In the present devices, an effective cross-section diameter of any part of the pore (e.g., nanoscale pore) is less than micron (1 μm), which for a circular cross-section would amount to a diameter less than 1000 nm. Thus, the effective cross-section diameter (or radius) may be an average diameter at a certain location if the pore is not circular in shape. Further, the aperture part inside the pore has an effective cross-section diameter, dA, less than 50 nm, and in some embodiments smaller than 50 nm for all its transverse cross-sections. However, the length of the pore is not restricted to nanoscale lengths. Any cross-section of the lumen part is at least 50 nm. In embodiments, the area of at least 90% of the cross sections (2) of the lumen part (A_{L}), e.g., all of them, is much larger than the area of any cross section of the aperture part (A_{A}), such that A_{L}>2A_{A}, i.e. the area of at least 90% or any cross section of the lumen part is larger than twice the area of the largest aperture cross section. This may also be defined as that the largest cross-sectional area of at least 90% of the aperture part is smaller than half the area of the smallest cross sectional area taken across the 90% of the lumen part of the pore. Typically, the difference in size is even larger, such that the area of the majority, such as ¾ (i.e. >75%), of the lumen cross sectional areas are at least 4 times larger than minimum pore cross section of the aperture part (A_{L}>4A_{A}), or even 8 or 16 times larger or more than minimum pore cross section of the aperture part (A_{L}>8A_{A} or A_{L}>16A_{A}). When having multiple sub-lumens, each sub-lumen of the lumen part has larger dimensions (effective cross-section diameter, along length) than the aperture part, as defined above. For instance, in some embodiments, more than three quarters of the cross-section areas are at least 4× larger than the minimum pore cross section area of the aperture parts. In some example embodiments more than three quarters of the cross-section areas are at least 8× larger, or even 16× larger than the minimum pore cross section area of the aperture parts.

The area of the cross-section of the first reservoir near (e.g., at) the connection with the lumen part is at least 1 μm. This distinguishes the reservoir from the lumen. Typically, the cross-section of the first reservoir is at least 1 μm along its whole length.

The area of the cross-section of the first reservoir near (e.g., at) the connection with the aperture part is at least 50 nm. This distinguishes the reservoir from the aperture. Typically, the cross-section of the first reservoir is at least 50 nm along its whole length.

Further, in embodiments, the pore (e.g., nanoscale pore) is such that at the ends of the pore (e.g., nanoscale pore), the cross-section area shows an enlargement, larger than a factor of four. Thus, the cross-section area at each opening (5, 6) of the pore (e.g., nanoscale pore), A_{O}, may show an enlargement of the pore compared to an area inside the pore, A_{I}, such that at least A_{O}>4A_{I}.

An example of a pore shape is illustrated in FIG. 2, a schematic cross-section of a pore (7, 8) having a narrow aperture (8) at the top, and a wide lumen (7) part below, with an electrode area (11) surrounding the top part of the lumen facing the aperture. For a maximal signal, the resistance of the aperture should approximately equal the resistance of the lumen, where the resistive divider criterion is thus defined as R_{A}≈R_{L}. For maximal SNR, accounting for thermal white noise, the resistance of the aperture should be approximately double that of the resistance of the lumen because the signal amplitude to noise amplitude ratio (SNR) in that case can be written as

$$SNR = \sqrt{{( \frac{Δ {G}_{p}}{{G}_{p}} )}^{2} \frac{{{G}_{p} ( V )}^{2}}{4 kTB} \frac{{α}^{2}}{{( α + 1 )}^{3}}} .$$

The SNR as written in this equation shows a maximum for

$$α = \frac{{G}_{c}}{{G}_{p}} = 2.$$

Here G_{c}=1/R_{c} and Gₚ=1/Rₚ. ΔGₚ is the modulation of the aperture resistance when a target molecule passes by, V is the total voltage applied across the total pore. k is Boltzman's constant, T temperature and B bandwidth. The reason to have an asymmetry between the aperture and the lumen is that a small aperture gives rise to a high translocation signal and high resolution, while a large lumen gives no disturbing translocation signal and can fit a large electrode. Larger electrodes mean less noise, and large electrode capacitance makes a device less sensitive to parasitic interconnect capacitance, providing better coupling to a remote FET, but it should not be too large to maintain bandwidth.

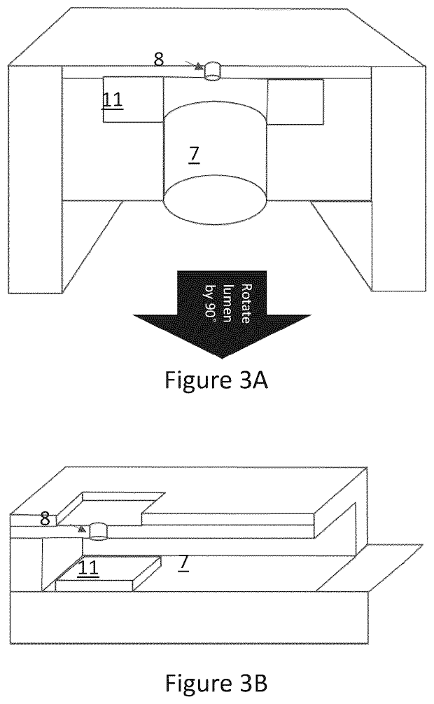

Example of schematic cross-sections of pore shapes are further illustrated in FIG. 3. In FIG. 3A a pore (e.g., nanoscale pore) (7, 8) is illustrated having an aperture (8) at the top, and a lumen (7) part below, with an electrode area (11) surrounding the top part of the lumen facing the aperture. The layer thickness determines electrode size, and the layer thickness cannot typically be varied on the same wafer. The electrode size is fixed by the effective lumen diameter, and to keep lumen resistance half of aperture resistance, the lumen depth is set by the lumen effective diameter. Varying effective diameter on the same wafer is hence difficult, as one cannot change thicknesses easily. The maximum electrode size is limited by the achievable vertical etch aspect ratio. By rotating the lumen in FIG. 3A 90°, one attains a pore as illustrated in FIG. 3B, where the lumen (7) and the aperture (8) are perpendicular instead of linear, thus attaining a lateral design. In this design, one may vary the electrode (11) size. Full electrode sizing freedom is attained since sizing is determined by layout, and in this case, it will also be easier to integrate and vary materials. This design also provides geometric freedom in lumen layout, as lumen size can be decoupled from electrode size. However, one needs to mind parasitic capacitance. In this design no aspect ratio limitations due to process (as geometry is mainly defined by mask) is present, nor are there any electrode size limitations due to the process. There is more freedom to shape the lumen, such as by mask design rather than by etch, and it is possible to obtain lots of different shapes on the same wafer.

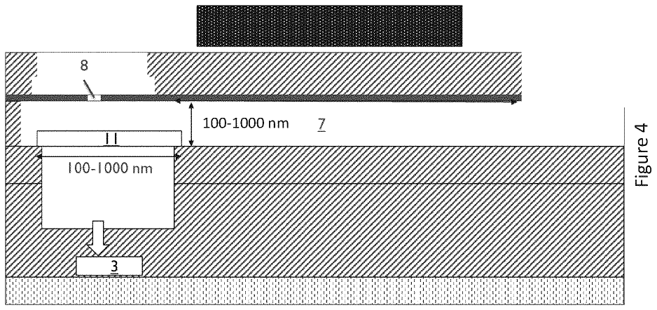

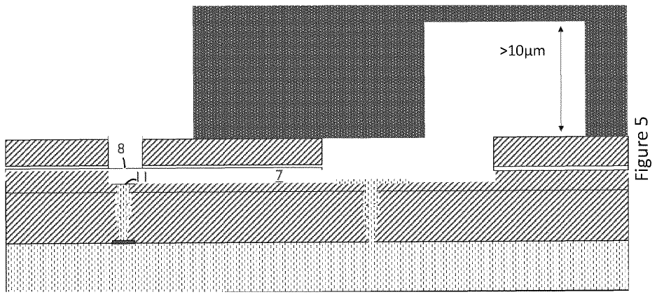

A further example of a lateral device design is illustrated in FIG. 4, showing the aperture (8), the lumen (7), a planar electrode (11) an insulator, a FET (3), a base of foundry Si (bottom layer), add-on processing on the foundry wafer (the whole structure minus the fluidics wall), and fluidics (top black rectangle) and a cis and trans reservoir and a pore (7, 8). FIG. 5 illustrates a further example of a lateral design, in a zoomed-out view of the cross section. The device comprising a foundry wafer (two first bottom layers), a cis reservoir in connection with an aperture (8), a sensing electrode (11), a bias electrode (shown on the right side of the sensing electrode), and a large channel (e.g. height >30 μm) to a trans reservoir, wherein the cis reservoir and the trans reservoir are separated by bonded glass or Si (black structure). FIG. 6 illustrates a further example of a lateral design similar to FIG. 5, but comprising multiple sub-lumens (7a, 7b).

The device typically comprises a wall/walls having a width/thickness w surrounding the nanopore. The area and the thicknesses of the insulating walls surrounding the pore (e.g., nanoscale pore) are such that the total summed capacitances between the electrolyte inside the pore and the outside electrical conductors, such as the first and second reservoirs and including the electrode, is smaller than 50 fF, smaller than 10 fF, or smaller than 1 fF.

In some embodiments, the geometry of the nanoscale pore is such that 1) if the pore and reservoirs are uniformly filled (in simulation) with a single isotropic and uniformly conducting test material or test liquid and 2) if it is made sure (in experiment or theory) that the main electrode makes electrical contact with this test material or liquid with negligible contact resistance (by removing any thin dielectric on the electrode) and negligible electrode resistance, then the (test) resistance between the aperture reservoir as electrically contacted by that reservoir's electrode and the main electrode (R1) approximately equals the (test) resistance between the lumen reservoir as electrically contacted by that reservoir's electrode and the main electrode (R2). Approximately equal resistance here refers to equal within a factor of ⅙ to 6 or within a factor of ⅓ to 3. In other words, R1/R2 may be from ⅙ to 6 or ⅓ to 3.

In some embodiments, the geometry of the pore (e.g., nanoscale pore) is such that 1) if the pore and reservoirs are uniformly filled (in simulation) with a single isotropic and uniformly conducting test material or test liquid and 2) if it is made sure (in experiment or theory) that the main electrode makes electrical contact with this test material or liquid with negligible contact resistance (by removing any thin dielectric on the electrode) and negligible electrode resistance, then the (test) resistance between the aperture reservoir as electrically contacted by that reservoir's electrode and the main electrode (R_{A}) approximately equals double the (test) resistance between the lumen reservoir as electrically contacted by that reservoir's electrode and the main electrode (R_{L}). Approximately double resistance here refers to equal within a factor of 0.27 to 23 or within a factor of 0.57 to 8.6. In other words, R_{A}/R_{L} may be from 0.27 to 23 or 0.57 to 8.6.

In other embodiments, the geometry of the pore is such that if the pore and reservoirs are uniformly filled (in experiment or theory) with a single isotropic and uniformly conducting test material or test liquid and 2) if it is made sure in experiment or theory that the main electrode makes electrical contact with this test material or liquid with negligible contact resistance (by removing any thin dielectric on the electrode) and negligible electrode resistance, then the lumen test resistance (R_{L}) approximately equals theta/2 (θ/2) times the aperture test resistance (R_{A}), i.e. θ/2*R_{A}. Approximately here refers to θ*R_{A}/R_{L} being within the range of 0.27 to 23 or within a range of 0.57 to 8.6. Theta (θ) is the resistivity of the electrolyte of the aperture reservoir/second reservoir (the nanopore device is meant to be used with) Re1, divided by the resistivity of the electrolyte of the lumen reservoir (the nanopore device is meant to be used with) Re2, i.e.

$$θ = \frac{Re 2}{Re 1} .$$

### [Equation 2]

For use with symmetric electrolytes theta equals one.

In some embodiments, the resistance of the lumen part, R_{L}, is approximately half the resistance of the aperture part, R_{A}, when no molecule is translocating through the pore, such that R_{A}~2R_{L}. R_{L} may be described approximately as

$${R}_{L} ∝ \frac{1 / 4}{rtop} + {\int }_{electrode top}^{top} \frac{1}{A ( l )} dl , \qquad [ Equation 3 ]$$

and R_{A} may be described approximately as

$${R}_{A} ∝ {\int }_{bottom}^{electrode bottom} \frac{1}{A ( l )} dl + \frac{1 / 4}{rbottom} . \qquad [ Equation 4 ]$$

Thus, the demanded proportion between the resistances may be written as:

$$F \frac{1 / 4}{rtop} + F {\int }_{electrode top}^{top} \frac{1}{A ( l )} dl = {\int }_{bottom}^{electrode bottom} \frac{1}{A ( l )} dl + \frac{1 / 4}{rbottom} , \qquad [ Equation 5 ]$$

i.e. the resistive divider condition of the geometry independent of electrolyte.
The total resistance may be approximately defined as:

$$R = ρ ( \frac{1 / 4}{rtop} + {\int }_{electrode top}^{top} \frac{1}{A ( l )} dl + {\int }_{bottom}^{electrode bottom} \frac{1}{A ( l )} dl + \frac{1 / 4}{rbottom} ) , \qquad [ Equation 6 ]$$

where ρ is the resistivity.

A(l) is the area of the cross section at position along pore axis 1, and top, bottom, electrode bottom and electrode top correspond to positions along the pore axis, where electrode refers to the main electrode. rtop and rbottom are the effective cross-section radii at the ends of the pore. F is the demanded proportion between resistances, F ranges from 0.27 to 23, or from 0.57 to 8.6.

Further, the pore part above the main electrode will have approximately half the resistance of the pore part below the main electrode, where the pore above and below the electrical center (9) may correspond to the lumen (7) and aperture (8) part, or may be partially overlapping as the main electrode may be partially situated in the different parts as discussed above, thus the pore above and below the main electrode is not necessarily the same as pore lumen part and pore aperture part, in which the resistance of pore below the electrode may be referred to as Rp (which may be identical to R_{A}, and the resistance of the pore above the electrode may be referred to as Rc (which may be identical to R_{L}). Typically, the largest part (>75%) of the area of the main electrode is exposed to the lumen part of the pore (the wide part of the pore). A pore where the pore part above and pore part below the (main) electrode have approximately double resistance may be realized in different ways. It can for example be translated in purely geometric criterion independent of electrolyte. The electrode satisfying this equal resistance criterion most closely is the main electrode, others are optional auxiliary electrodes. It should be noted that an electrode does not need to entirely wrap around pore. The electrode is typically made of a conductive material, which may be selected from (but not limited to) the following materials: titanium nitride, TiN, ruthenium, Ru or platinum, Pt, or silicon, Si. The electrode can for example be covered with a thin dielectric.

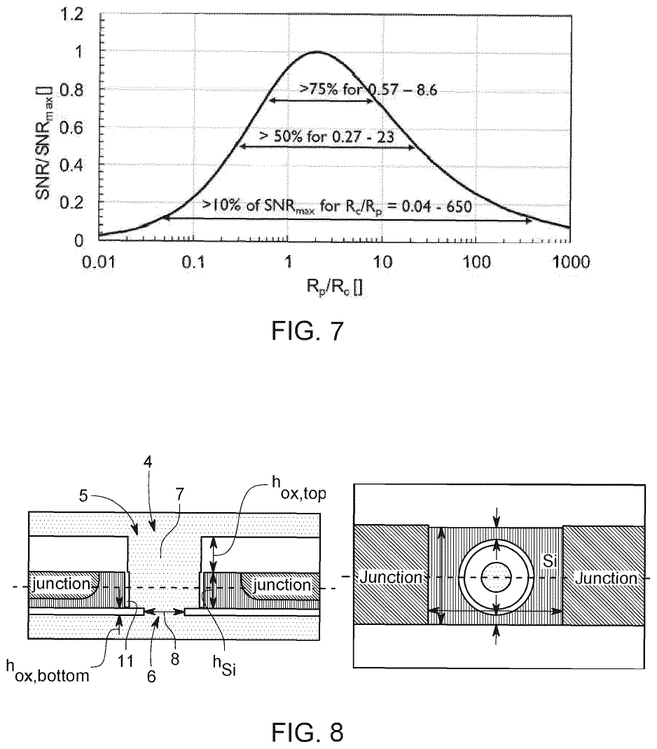

That the resistance is “approximately” double could for example be defined as meaning that R_{c}/Rₚ can be in a range of 0.04-650, this would imply a reduction by at most a factor of 1/10 versus the improved signal to noise ratio (SNR>10% of the improved value). In another example, R_{c}/Rₚ can be in a range of 0.27-23 which would imply a reduction by at most a factor of ½ versus the improved signal to noise ratio (SNR>50% of the improved value), or in a third example, R_{c}/Rₚ can be in a range of 0.57-8.6 which would imply a reduction by at most a factor of ¾ of the improved signal to noise ratio (SNR>75% of improved SNR). Approximately double the resistance may refer here to being in a range of 0.27-23 or within a range of 0.57-8.6. This is illustrated in FIG. 7, where the relation between the ratio of resistances and the improved signal to noise ratio is shown, such that ×½ of improved signal modulation corresponds to 0.27-23 leeway in R_{c}/Rₚ, and similarly that ×¾ of improved signal modulation corresponds to 0.57-8.6 leeway in R_{c}/Rₚ.

It should be appreciated that the lumen part is so large, the height or length of the lumen part of the nanopore can be larger than 1_micron, such as multiple microns, or multiple 10s of microns, or larger, and the cross sections is so large, but typically smaller than 1 micron in effective diameter, that when a target molecule passes through, no significant modulation of the resistance (R_{L}) occurs, while the aperture part is so narrow, the height being 50 nm or smaller and the effective diameter being 20 nm or smaller, that when a target molecule passes through, a significant modulation of the resistance (R_{A}) occurs. The lumen part may have a uniform shape (typically of equal width) or may be tapered, becoming smaller and smaller when approaching the aperture, to relax requirements on the top part. Thus, it should be realized that the lumen part of the pore need not necessarily have a uniform cross-section, but the pore may have a shape of a truncated cone, such that the pore provides a minimum cross-section at one end of the pore and a maximum cross-section at an opposite end of the pore.

In FIG. 8 a nanopore-FET device is illustrated according to an embodiment, wherein the nanopore (4) is embedded in the FET, such that the FET is gated by an electrolyte-filled nanopore running through the channel region of the FET, where FIG. 8 (left) illustrates a side view of the device/pore, and FIG. 8 (right) illustrates a top view. The device comprises a semiconductor wall having a width/thickness w surrounding the nanopore, where the wall thickness w is 10 nm or less, such as 5 nm or less, which wall thickness is illustrated as Si wall thickness in FIG. 8 (right). Wall width/thickness is the width of the electrode/channel between lumen and opposing channel edge. For example, the Si wall thickness is the width of the silicon channel formed at the narrowest constriction between the pore and sidewall passivation. As illustrated in FIG. 8 (right), the wall comprises at least three different layers comprising at least two different materials, the layers being arranged on top of each other such that a dielectric material is arranged at the top and bottom of the pore, the top dielectric layer having a height h_{ox_top} and the bottom dielectric layer having a height h_{ox_bottom}, and a conductive/semi-conductive material (11) is arranged in the middle of the pore and having a height hₛᵢ, the semiconductor material typically being silicon (Si) and having a height defined as hₛᵢ of 5-100 nm, or more. It may thus be seen that in this example, the height of the lumen part of the pore, h_{L}, is defined by h_{L}=h_{ox_top}+hₛᵢ, wherein h_{L} is in the range of 50 nm-5 μm, and the height of the aperture part of the pore, h_{A}, is defined by h_{A}=h_{ox_bottom}, wherein h_{A} is typically less than 50 nm. The effective diameters of the lumen part and aperture part (i.e. the measurement of the pore parallel to the cross sections shown in FIG. 1, where the radius, r, of the cross sections relate to the effective diameter, d, such that 2r=d, and the effective diameter is perpendicular to the height of the lumen and aperture parts) relate such that the area of any cross section of the lumen part is at least twice the area of the smallest area of any cross section of the aperture part, as defined above. The effective diameter of the lumen part, d_{L} is in the range of 50-300 nm, and the effective diameter of the aperture part, d_{A} is less than 20 nm. The semiconductor wall width and the FET width from a top view of the device/pore are illustrated in FIG. 8 (right).

The demands on the device for realizing a high signal readout with low noise, include that it should attain a negligible signal from the lumen part of the pore, which is attained by the majority of the lumen part of the pore being wide, and that the area of cross sections of the lumen part should be much larger than the aperture part, such as typically 4 or even 8 times larger, as described above. A second demand, to attain the high signal and high resolution, is that the aperture part of the pore should be small, such as being short (a low height h_{A}) and narrow (a small effective diameter d_{A} and thus a small area A_{A} of a cross section of the aperture part). The signal strength may be independent of the resolution as defined by

$$Δ V \approx \frac{Δ G}{G} ∝ \frac{1}{h A} \frac{hA}{AA} = \frac{1}{AA} , \qquad [ Equation 7 ]$$

where h_{A} is the height (length) of the aperture part, and A_{A} is the area of an aperture cross section. Thus, it may be seen that the resolution corresponds approximately to the height/length of the aperture, i.e. resolution=h_{A}. Thus, the size of the aperture may be adapted based on the application of the nanopore FET device, such that desired resolution and limits are met, where the size of the aperture opening (6), i.e. the effective diameter of the aperture, is typically sub 50 nm, such as sub 20 nm. A third demand on the device is the resistive divider criterion, to obtain the highest signal or signal to noise ratio for the overall pore, such that the resistance of the lumen part is approximately the same as the aperture part for maximal signal and such that the resistance of the lumen part is approximately half that of the aperture part for maximal signal-to-noise ratio.

The demands above are structural demands, wide pore in lumen part (or part above electrical center) and narrow and short pore in the aperture part (or part below electrical center), which are independent of electrolyte. Assuming a uniform electrolyte, and as we aim for resistance proportions 2R_{L}=R_{A} or Rp=2Rc, which corresponds approximately to

$$2 \frac{1 / 4}{rtop} + 2 {\int }_{electrode top}^{top} \frac{1}{A ( l )} dl = 1 / 4 l + \frac{1 / 4}{rbottom}$$

(equation 5), resistivity drops out of the equation, resulting in a purely geometric definition. The criterion would be the same if the empty space is filled with any material of uniform conductivity. Here, the surface charge is not accounted for. To account for surface charge, the definition can be altered, where a parameter can be introduced which depends on electrolyte conditions. It could be noted that surface charge is non-ideal, which is sought to be avoided. Surface charge matters less at the typical high salinities used for nanopores.

In the case of asymmetric electrolyte, the resistivity ρ of cis and trans reservoir electrolyte is different, which may be characterized by an asymmetry factor theta

$$θ = \frac{{ρ}_{1}}{{ρ}_{2}}$$

### [Equation 8].

The demands on wide lumen part of the pore (or part above electrical center) and narrow and short aperture part of the pore (or part below electrical center), are independent of electrolyte still, but for the 2R_{L}=R_{A} or Rp=2Rc, the resistivity does not drop out of the equation, and we thus introduce the asymmetry factor theta;

$$2 \frac{1 / 4}{rtop} + 2 {\int }_{electrode top}^{top} \frac{1}{A ( l )} dl = \frac{1}{θ} ( {\int }_{bottom}^{electrode bottom} \frac{1}{A ( l )} dl + \frac{1 / 4}{rbottom} ) , \qquad [ Equation 9 ]$$

where the criterion would be the same if empty space is filled with any material with uniform conductivity, adjusting the criterion to θR_{A}=2R_{L}.

Thus, the nanopore of the FET-based nanopore device consists of two parts, an aperture with a small effective diameter and low height as well as a lumen with larger effective diameter and height. The aperture with smaller effective diameter is defined in a dielectric layer below the silicon semiconductor layer. The thickness of the aperture dielectric layer is reduced in order to increase the resolution with which features can be resolved on a translocating molecule. For improved signal magnitude the thickness of the bottom insulator is may be of a similar thickness (or somewhat smaller) as the molecular feature to be detected. The aperture effective diameter may be as small as possible. The lumen with larger diameter is defined in the silicon layer and an insulator layer on top of the silicon. The silicon layer may be between 5 and 100 nm thick. The top oxide thickness is chosen to obtain an improved signal magnitude, which is obtained for an improved resistive divider condition, where the electrolyte in the top insulator part or lumen part (including access and spreading resistances) should have equal resistance as the electrolyte in the aperture part (including access and spreading resistances). In the devices of the present disclosure, the effective diameter of the lumen is chosen larger than the effective diameter of the aperture resulting in an “asymmetric device” (large lumen, small aperture), in order to still allow for an improved signal magnitude while allowing a thicker silicon or electrode layer (5-100 nm). The lumen part of the nanopore may have a uniform shape (substantially equal effective diameter throughout) or a tapered shape. The relation between the sizes of the lumen and aperture parts are essential, as this enables the larger bandwidth and low noise. The present design allows one to decouple the FET design in the lumen from the aperture which determines the translocation signal. In this way the FET can be made larger to control the short channel effects, while still maintaining a sufficient translocation signal, because the aperture can be made very small without affecting FET properties. The FET active area determines the bandwidth but can still be kept sufficiently small to realize bandwidths >1-10 MHz. Hence, the present devices allow a high-quality FET with suppressed short channel effects, a sufficient signal magnitude and resolution nanopore detector and high bandwidths (>1-10 MHz). Thus, the devices of the present disclosure may operate with bandwidths over 100 kHz, or even with bandwidths higher than 1 MHz due to nanoscale cross-sections of fluidic passage.

To obtain sufficiently suppressed short channel effects, different designs of the device may be beneficial. For example, the nanopore effective diameter in the lumen part (silicon region) should not be made too small, such that it may be beneficial to maintain a pore effective diameter larger than 10 nm, or in some embodiments larger than 20 nm. Typically increasing nanopore effective diameter decreases signal strength. This is avoided here by providing the narrower ‘aperture’ pore in the bottom insulator. This aperture will determine signal strength. Thus, by combining a larger lumen which can house a larger main electrode with a small aperture, negative effects to the signal strength may be mitigated. Further, it may be beneficial to reduce the (silicon) wall thickness. For example, the wall thickness is the width of the silicon channel formed at the narrowest constriction between pore and sidewall passivation. This semiconductor wall thickness could be reduced below 10 nm, or in some embodiments below 5 nm. In addition, thinning down the silicon of the FET further suppresses short channel effects. Further, it should be considered that short channel effects depend on the channel length of the FET device (the distance between source and drain junctions) and the effects become worse for decreasing gate length. Hence, the distance of the source and drain junctions may be adjusted to regulate the short channel effects.

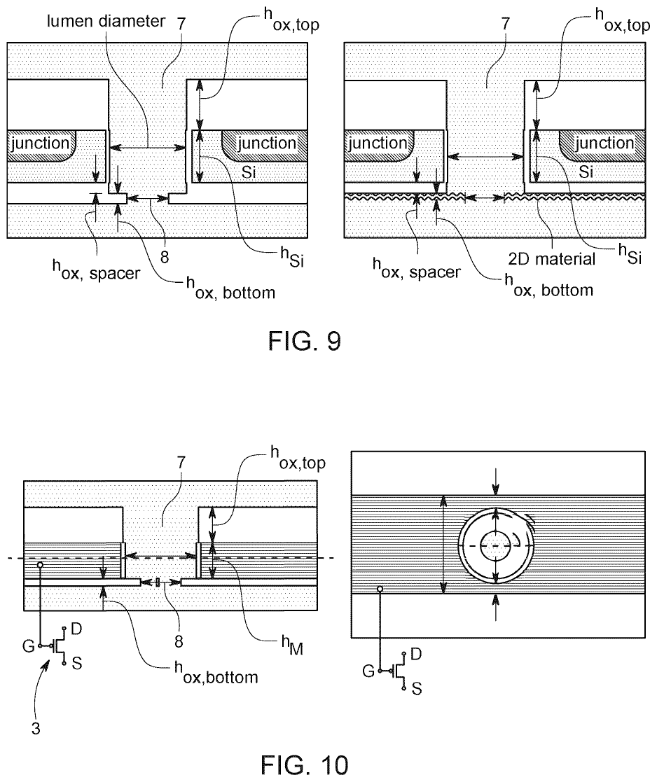

As described above in relation to FIG. 3, in an embodiment the FET may be embedded in the nanopore, and the aperture has a height corresponding to the height of the bottom layer. In view of this, different designs may be possible. One variation allows for a thicker dielectric on the bottom of the silicon to prevent shunting currents running in the bottom of the silicon. Another variation makes use of a 2D material (e.g., boron nitride, graphene, dichalcogenide) as the aperture material to realize the thinnest possible aperture membrane for very high molecular resolution, as illustrated in FIG. 9. A fourth layer may be arranged below the bottom dielectric material and have a heigh h₄, wherein h_{L}=h_{ox_top}+hₛᵢ+h_{ox_bottom} and h_{A}=h₄. As illustrated in FIG. 9 (left), introduction of a dielectric multi-layer consisting of multiple different dielectric materials allows for a thicker dielectric on the bottom of the (silicon) wall while still having a thin aperture height (hₐₚₑᵣₜᵤᵣₑ, h_{A}). This may allow for suppressing shunt currents on the bottom of the Si, which grow larger with thinner oxides on the bottom of the silicon. The thin aperture height allows a higher resolution for resolving closely spaced features on target molecules. As illustrated in FIG. 9 (right), introducing a 2D material (as discussed above) below the bottom oxide allows for an atomically thin aperture layer (low h_{A}) for which a very high resolution to resolve closely spaced features on molecules is attained.

In an alternative embodiment, an extended gate variation of the present device is presented, where the silicon in the pore is replaced with an electrode material, wherein the device comprises a remote, extended gate FET, where an electrode wrapped around the nanopore is connected to a remote FET. The electrode material is typically a metal, such as titanium nitride, TiN, ruthenium, Ru or platinum, Pt, which is coupled to the gate of a silicon transistor remote from the pore. The design allows a sufficiently large metal-electrolyte surface area of the electrode material in the lumen and hence a larger capacitance of the electrode. This allows to limit the signal reduction due to the coupling to a readout transistor, which may be implemented in relatively standard CMOS technology. A standard CMOS FET typically has a larger capacitance than an aggressively scaled device. Having an increased electrode capacitance leads to a reduced signal decrease due to the capacitive divider which determines signal transfer between electrode and remote FET. A larger electrode with larger capacitance also leads to less signal reduction due to the interconnect capacitance due to the electrode-to-FET interconnect. More standard CMOS technology may be used due to cost reasons, and also because analog design utilizes larger FETs to suppress noise and variability.

FIG. 10 illustrates an example of the remote gate sensor embodiment, FIG. 10 (left) illustrating a side view of the device/pore, and 10 (right) illustrating a top view. In the schematic cross-sections of the device design for an extended gate pore FET, the metal electrode is connected to the gate of a nearby FET (3) implemented in standard CMOS technology. Compared to FIG. 8, the middle layer, hᵢₛ, in this embodiment is made from a metal is illustrated in FIG. 10 (right, short vertical double arrows at the top and bottom of the circle).

The two main embodiments, the embedded FET (silicon pore-FET) and the remote FET (extended gate pore-FET) have several main characteristics in common. By increasing the lumen size and the active area, the noise is decreased. The larger lumen does not affect the signal (which is caused by the molecule in the aperture). The lumen resistance approximately equals half the aperture resistance. The resistance of the lumen should be approximately half the resistance of the aperture region. Or the resistance of the region above the electrical center should be half that of the resistance of the region below the electrical center. The aperture effective diameter and height are kept low to enhance signal magnitude and resolution, respectively. Hence, noise reduction with maintained signal means the Signal-to-Noise Ratio (SNR) is improved when enlarging the lumen. Furthermore, the capacitance associated with the active area, determines the bandwidth limit due to the electrode-to-electrolyte capacitance. When enlarging the lumen to enhance SNR, the lumen can still be kept sufficiently small to allow the SNR gains but also realize bandwidths above 1-10 MHz.

Also, for the embedded FET, due to the design (respective size difference between the lumen and aperture) the FET can be made larger to control the short channel effects of the silicon nanopore-FET. The larger nanopore FET also entails lower FET noise, and hence higher SNR.

For the extended gate nanopore-FET embodiment, due to the design one can have a large metal-electrolyte surface area of the electrode material in the lumen and this means a larger capacitance of the electrode. The larger capacitance allows to strongly limit the signal reduction due to the capacitive coupling to the readout FET. Moreover, this also allows to choose a larger readout transistor as typically seen in analog technology, and for cost reasons.

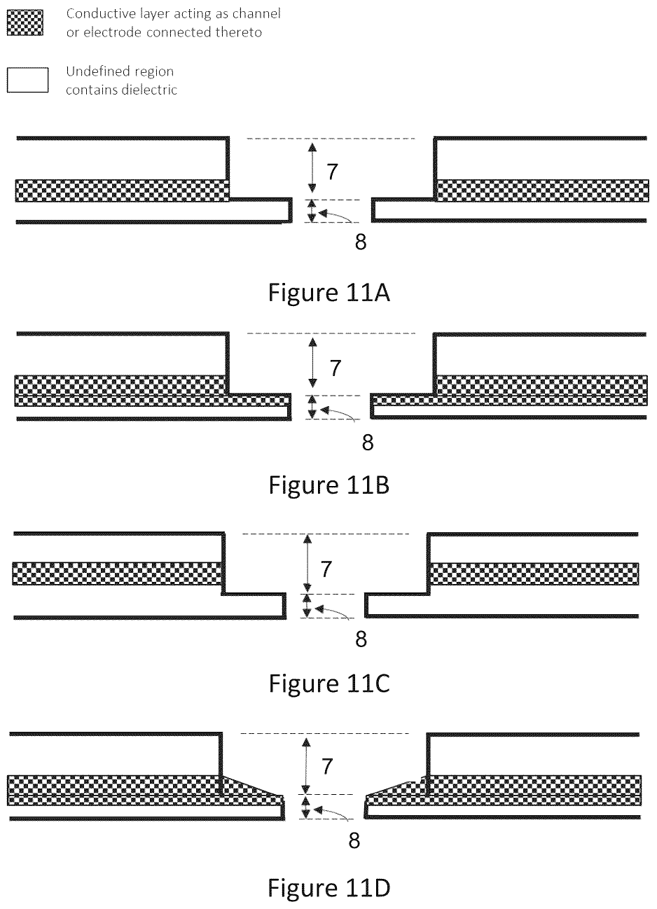

FIG. 11 illustrates some possible alternative designs of the present devices. FIG. 11A illustrates the general concept having a first part of the pore, a lumen part (7), and a second part of the pore, an aperture part (8), where a conductive material, acting as channel or having an electrode connected thereto, is present in the lower part of the lumen part. In FIG. 11B a first variation of the concept is illustrated, where parts of the pore are the same, but the main electrode is located both in the lumen (first) part and aperture (second) part of the pore. In FIG. 11C, a second variation of the main concept is illustrated, where the conductive material, acting as channel or having an electrode connected thereto, is present in the lower part of the lumen part, but with a spacer between the main electrode and the aperture part of the pore. In FIG. 11D, a third variation of the main concept is illustrated, where a slanted/sloped main electrode is used.

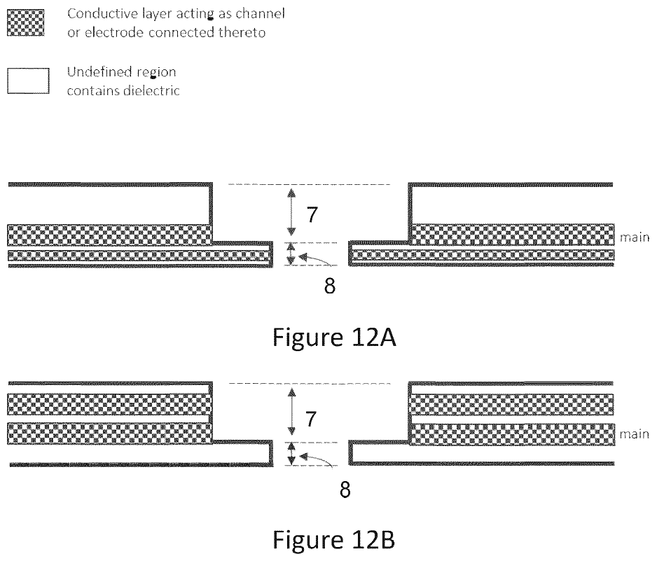

Besides the main electrode, a plurality of additional electrodes may be used, such as one or two additional electrodes besides the main electrode, thus the apparatus may have multiple sensor regions. As illustrated in FIG. 12A, depicting a first cavity (lumen) (7), and a second cavity (aperture) (8), where a conductive layer, acting as channel or having an electrode connected thereto, is present in the lower part of the first cavity, and in addition, a second electrode in present in the second cavity. Thus, it is possible to measure the change in resistance in different parts directly. As an alternative, as illustrated in FIG. 12B, both the main electrode and the additional electrode is located in the first cavity, thus enabling direct charge sensing.

A nanopore Field-effect transistor (FET) device is provided, comprising a nanopore/nanopore sensor and a FET. The nanopore sensor of the present disclosure may comprise a solid state nanopore. In some embodiments, the nanopore is embedded in the FET, such that the FET is gated by an electrolyte-filled nanopore running through the channel region of the FET. In other embodiments, the device comprises a remote FET, where an electrode wrapped around the nanopore is connected to the remote FET. A system comprising the device may also be provided. The system may comprise a first and second reservoir, optionally comprising an electrically conducting fluid, such as an electrolyte, a nanopore sensor device, as described above and below, comprising a nanopore connecting the two reservoirs. The target molecules/sample to be analyzed may be present in the first reservoir, and may during operation of the device exit the first reservoir, enter the lumen of the nanopore of the device, translocate through the nanopore along the pore axis and exit through the aperture into the second reservoir, wherein the lumen/electrolyte in the lumen has a resistance R_{L} and the aperture/electrolyte in the aperture has a resistance R_{A}, and the aperture has a size such that a significant modulation of the resistance R_{A} occurs when a target molecule translocates through the aperture, while the lumen has a size such that no significant modulation of the resistance R_{L} occurs when a target molecule translocates through the lumen.

The pore (e.g., nanopore) sensor may comprise a first and second electrolyte reservoir, respectively, being separated by a barrier comprising a nanopore. The sensor/system may further comprise electrodes for translocating molecules through the nanopore from the first electrolyte reservoir to the second electrolyte reservoir, wherein at least one of the first and second electrolyte reservoirs comprises the target molecule. The pore (e.g., nanopore) may have an aperture through which the molecule is translocated. As pores are not necessarily circular, the term “effective diameter” of the pore (e.g., nanopore) at a particular location refers to the average diameter of the pore at that location.

According to an embodiment, the resistance of the pore parts may be tuned. This may be performed by adjusting the size of the said-parts. Alternatively, the carrier concentration of the electrically conductive fluid (e.g. a salt concentration of an electrolyte) may be adjusted. Alternatively, the surface charge may be adjusted, by controlling the pH, as discussed in more detail below. It was noted that the aperture and lumen resistance can be tuned by having a low surface charge shallow pore (aperture part) combined with a high surface charge high pore (lumen part).

In the present devices, it is possible to provide surface charges on a surface of the pore along the extension from the first side to the second side. Having surface charges on the surface of the pore implies that the fluid in the pore may exhibit charge carriers of an opposite sign near the pore walls and increase the conductance of a certain part of the pore. According to an embodiment, a concentration of the surface charges is in a range of 1×10¹²-1×10¹⁵ cm⁻². Tuning surface charges can be used to tune the resistance of the pore or parts of it.

Surface charges may be formed on the surface of the pore by (de)-ionization of OH-groups or other ionizable groups at the surface of the thin dielectric layer. This may be caused by exposing the pore FET to the electrolyte fluid. For instance, if silicon dioxide (SiO₂) is used as the thin dielectric layer, depending on pH, silanol groups ionize when the silicon dioxide is immersed in an aqueous solution (which may be used as the electrolyte fluid), and ionization of silanol molecules at the surface of the thin dielectric layer would form negative surface charges on the surface of the pore for a neutral (pH=7) solution. Charge density may have a dependence on pH value of the electrolyte fluid, such that control of pH value may provide a control of concentration and sign of the surface charges.

If the surface charge concentration is very high, the surface charges could cause a high ion concentration in the fluid in the pore, such that an impact of the charge of the particle to be detected on the effective gate voltage may be reduced and, hence, the modulation of the electrical potential distribution in the pore by the particle to be detected may be reduced. Therefore, the surface charge concentration should not be too high.

In some aspects simulation methods are further provided for designing a pore Field-effect transistor (FET) device according to the present disclosure, where the measurements of the device are tuned/adapted to attain desired resistance properties.

As discussed above, a smaller lumen with smaller electrode surface allows higher bandwidth, and a certain bandwidth target determines a ceiling for lumen radius and silicon or electrode area. However, SNR favors increasing lumen size.

To test the implications of the properties on the bandwidth and the SNR, a device was constructed in which the lumen radius was 40 nm and the Si thickness which is identical to the electrode height in this case is 100 nm as in the device illustrated in FIG. 9 or 10. It was observed upon simulation that the bandwidth is still above 100 MHz. It was thus concluded that the lumen can still be considerably large while still allowing high bandwidth. For the processing in this particular example, a smaller lumen radius reduces the height-to-diameter aspect ratio of the lumen—AR becomes smaller for smaller radius, and higher aspect ratios are more difficult to realize/process.

Noise decreases with increased lumen radius. This dependence is mainly due to FET 1/f noise and much less due to the noise of the electrolyte-filled nanopore itself. This means that if FET 1/f noise is low vs. other noise sources (e.g. for foundry quality FETs) lumen radius will have limited impact on noise. For low 1/f noise, there is still a considerable SNR improvement when scaling the lumen radius up to ~20 nm since signal is boosted with increasing lumen radius considerably for radii up to ~20 nm. For good quality FETs (with medium noise), noise is reduced considerably when increasing lumen effective diameter up to ~20-40 nm (which is determined by the relevance of FET 1/f noise vs. electrolyte noise). Beyond 20-40 nm limited reductions in noise are obtained for this particular example.

The SNR grows with the square root of the Si (or electrode) thickness. This is due to a decrease in noise. The dependence of signal on Si thickness is weak. There is no SNR improvement when making silicon thin. A nanopore design, where the lumen radius is large vs. the aperture radius, results in high resolution because of the thin aperture. There is no strong lumen geometry dependence of the resolution for the asymmetric design, as the resolution depends on the aperture.

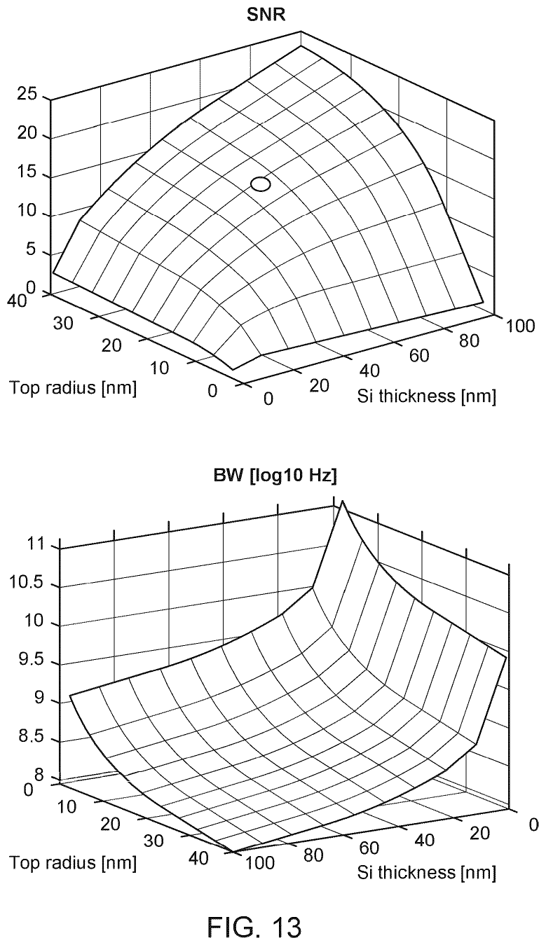

To determine the impact of the size of the lumen part radius (top radius) and the Si thickness in the devices of the present disclosure, different designs were tested and the results summarized in FIG. 13, illustrating the pore bandwidth (BW) and SNR trade-off. FIG. 13 (left) shows the impact of the design measures on the SNR, while FIG. 13 (right) illustrates the impact of the design measures on the BW. As shown, nanoscaling is driven by bandwidth, with SNR gains for larger devices becoming limited. Processing considerations also favor smaller effective lumen diameter in this particular design, and lumen aspect ratio becomes difficult for larger radius.

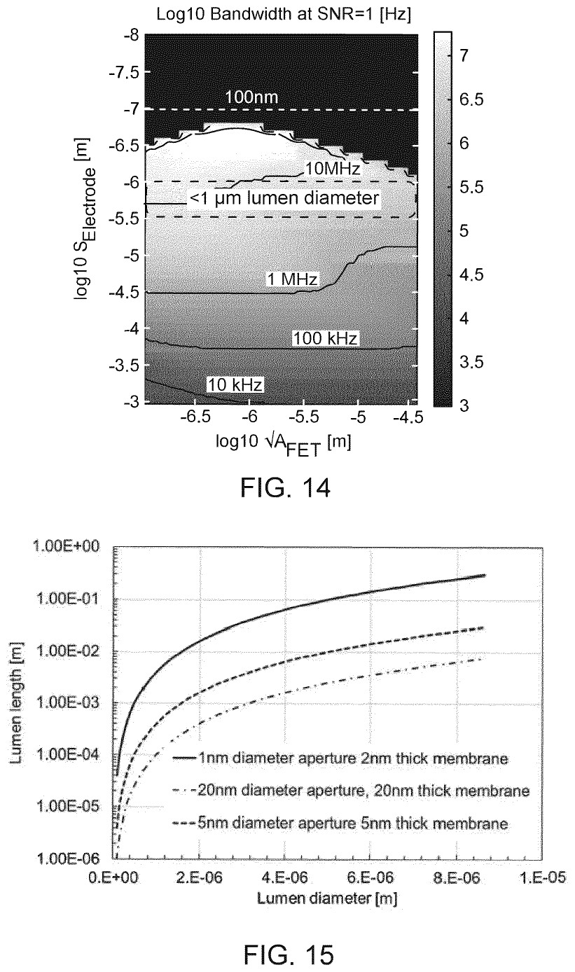

The main electrode area is key to obtain the desired SNR and bandwidth properties. A simulation of the bandwidth obtained for which SNR>1 is plotted in FIG. 14 as a function of electrode side S_{electrode} and the square root of FET gate area, A_{FET}. The electrode is assumed square for this simulation with area S_{electrode}². Based on this graph it becomes clear that in some example embodiments the electrode area is larger than 100×100 nm (10000 nm²). Around that size and smaller sizes SNR drops below 1 and hence negligible bandwidth with SNR>1 is obtained. Bandwidths of 10 MHz are obtained with electrode side ~<1 micron illustrating the bandwidth benefit of the pore.

To illustrate why increasing the lumen diameter beyond 1 micron is not desirable FIG. 15 plots the lumen length versus lumen diameter for large lumen diameters in the order of microns. As lumen diameter increases above 1 micron the lumen length for a flow-through lumen as illustrated in FIG. 6, the lumen reaches lengths in the order of millimeters, particularly for highly resistive, small apertures. Such lengths will not allow to integrate such pores in high numbers in the order of tens of thousands, or hundreds of thousands or millions on a single chip and are hence not desirable. A single-ended horizontal lumen would be four times shorter, but the lumen length increases when scaling diameter above 1 micron would still be undesirable for scaling up the number of pore components per chip. Similarly vertical lumens of such length are undesirable to integrate on chip.

1. A pore Field-effect transistor (FET) device for sensing of target molecules in an electrolyte, the device comprising:
a pore having at least a first opening and a second opening and comprising two contiguous parts, a lumen part and an aperture part, such that the lumen part is fluidically connected at least to the first opening and the aperture part is fluidically connected to the second opening;
at least a first reservoir and a second reservoir, the pore being fluidically connected to the first and second reservoirs, wherein the first reservoir is in connection with the lumen part of the pore via the first opening, and wherein the second reservoir is in connection with the aperture part of the pore via the second opening;
a sensor region comprising a main electrode that overlaps at least one of the lumen part or the aperture part of the pore, such that a target molecule passing through the pore from the first opening to the second opening passes through the sensor region;
wherein a transverse cross-section of the pore has an area, an effective cross-section diameter of the transverse cross-section, and an effective cross-section radius of the transverse cross section that vary along the pore, and
wherein a geometry of the device is such that:
the effective cross-section diameter of every transverse cross-section of the pore is less than 1 μm;
the effective transverse cross-section diameter, d_{A}, of every transverse cross-section of the aperture part is less than 50 nm;
an area of at least one transverse cross section of the aperture part is less than half an area of at least one transverse cross section of the lumen part, wherein at least ¾ of the transverse cross sections of the lumen part have areas that are at least four times larger than the area of any transverse cross section of the aperture part; and
an area of the main electrode that is exposed to the electrolyte is larger than 10000 nm².

2. The device according to claim 1, wherein the geometry of the device is such that:
when the pore and the first and second reservoirs are filled with a conducting test material or test liquid and the main electrode makes electrical contact with the test material or test liquid,
R_{A}/R_{L} is from 0.27 to 23, wherein R_{A} is an electrical resistance between the main electrode and the second reservoir, which is fluidically connected to the aperture, and wherein R_{L} is a resistance between the main electrode and the first reservoir.

3. The device according to claim 1, wherein the geometry of the device is such that:
when the pore and the first and second reservoirs are each filled with respective conducting test materials or test liquids, first reservoir is filled with a test material or test liquid with resistivity Re1, and the second reservoir is filled with a test material or test liquid with resistivity Re2, and the main electrode makes electrical contact with a test material or test liquid,
a test resistance of the lumen part, R_{L}, between the main electrode and the first reservoir and a test resistance of the aperture part, R_{A}, between the main electrode and the second reservoir are related such that θ*R_{A}/R_{L} is from 0.27 to 23, wherein
$$θ = \frac{Re 2}{Re 1} ,$$

4. The device according to claim 1, wherein the pore part located above an electrical center on the lumen side has a resistance R_{c} and a pore part located below the electrical center on the aperture side has a resistance Rₚ, wherein a geometry of the device conforms to:
$$\frac{\frac{F}{4}}{rtop} + F {\int }_{electrode top}^{top} \frac{1}{A ( l )} dl = {\int }_{bottom}^{electrode bottom} \frac{1}{A ( l )} dl + \frac{\frac{1}{4}}{rbottom}$$
wherein A(l) is the area of the cross section at position l along the pore, wherein top, bottom, electrode bottom, and electrode top correspond to positions along the pore, where electrode refers to the main electrode, wherein rtop and rbottom are the effective cross-sectional radii at the ends of the pore,
wherein F is from 0.27 to 23, and wherein Rₚ/R_{c} is from 0.27 to 23.

5. The device according to claim 1, wherein the lumen part comprises a first sub-lumen and a second sub-lumen, wherein the first sub-lumen is fluidically connected to the first opening and the second sub-lumen is fluidically connected to a third opening, wherein the device further comprises a third reservoir, and wherein the third reservoir is in fluidic connection with the second sub-lumen of the pore via the third opening such that the pore is fluidically connected to the first, second and third reservoirs.

6. The device according to claim 1, wherein the main electrode is located on the walls of the pore and at least ¾ of the electrode area is exposed to the lumen part of the pore.

7. The device according to claim 1, wherein the electrode comprises a thin solid dielectric material present between a bulk material of the electrode and the space inside the pore.

8. The device according to claim 7, wherein the thin solid dielectric material is 0.5-10 nm thick and comprises Al₂O₃, TiO₂, ZrO₂, HfO₂, Si₃N₄, or SiO₂.

9. The device according to claim 1, wherein the main electrode comprises TiN, Ru, Pt, or silicon.

10. (canceled)

11. (canceled)

12. The device according to claim 1, wherein
the aperture part has a height h_{A} and an effective diameter d_{A}, and wherein h_{A} is less than 50 nm and d_{A} is less than 20 nm; and
the lumen part has an effective diameter d_{L} from 70 to 1000 nm.

13. (canceled)

14. The device according to claim 1, wherein the device further comprises a dielectric wall of thickness w surrounding the pore, wherein the thickness of the wall is such that the total summed capacitance between electrolyte inside the pore and outside electrical conductor that include the first and second reservoirs and the main electrode, is smaller than 50 fF.

15. The device according to claim 1, wherein the device further comprises a semiconductor wall of thickness w_{Si} surrounding the pore, wherein the wall thickness w_{Si} is 10 nm or less.

16. The device according to claim 14, wherein the dielectric wall comprises at least three different layers comprising at least two different materials, the layers being arranged on top of each other such that a dielectric material is arranged at the top and bottom of the pore, wherein the top dielectric layer has a height h_{ox_top} and the bottom dielectric layer has a height h_{ox_bottom}, wherein a conductive or semiconductive material is arranged in the middle of the pore, and wherein the middle layer has a height hₛᵢ.

17. (canceled)

18. (canceled)

19. The device according to claim 1, wherein the lumen part of the pore has a uniform shape or a tapered shape.

20. The device according to claim 1, wherein the device comprises one or more additional electrodes located in at least one of the aperture part or the lumen part of the pore.

21. The device according to claim 1, wherein the pore is embedded in the FET such that the pore runs through a channel region of the FET and further such that the FET is gated by an electrolyte-filled disposed within the pore, and wherein the sensor region comprises a source-drain axis.

22. (canceled)

23. The device according to claim 21, wherein a fourth layer is arranged below the bottom dielectric material and has a height h₄, wherein h_{L}=h_{ox_top}+hₛᵢ+h_{ox_bottom} and h_{A}=h₄, and wherein h_{L} is a height of the lumen and h_{A} is a height of the aperture.

24. (canceled)

25. (canceled)

26. The device according to claim 1, wherein the device comprises a remote extended FET, where an electrode wrapped around the pore is connected to a remote gate sensor.

27. The device according to claim 16, wherein the dielectric wall comprises at least one of titanium nitride, TiN, ruthenium, Ru, or platinum, Pt, which is coupled to a gate of a silicon transistor remote from the pore.

28. A system for sensing of target molecules in an electrolyte, the system comprising:
a first reservoir comprising target molecules in an electrolyte;
a second reservoir;
a pore Field-effect transistor (FET) device, comprising:
a pore having at least a first opening and a second opening and comprising two contiguous parts, a lumen part and an aperture part, such that the lumen part is fluidically connected at least to the first opening and the aperture part is fluidically connected to the second opening;
wherein the pore is fluidically connected to the first and second reservoirs, wherein the first reservoir is in connection with the lumen part of the pore via the first opening, and wherein the second reservoir is in connection with the aperture part of the pore via the second opening;
a sensor region comprising a main electrode that overlaps at least one of the lumen part or the aperture part of the pore, such that a target molecule passing through the pore from the first opening to the second opening passes through the sensor region;
wherein a transverse cross-section of the pore has an area, an effective cross-section diameter of the transverse cross-section, and an effective cross-section radius of the transverse cross section that vary along the pore, and
wherein a geometry of the device is such that:
the effective cross-section diameter of every transverse cross-section of the pore is less than 1 m;
the effective transverse cross-section diameter, d_{A}, of every transverse cross-section of the aperture part is less than 50 nm;
an area of at least one transverse cross section of the aperture part is less than half an area of at least one transverse cross section of the lumen part, wherein at least ¾ of the transverse cross sections of the lumen part have areas that are at least four times larger than the area of any transverse cross section of the aperture part; and
an area of the main electrode that is exposed to the electrolyte is larger than 10000 nm²;
wherein the sensing of target molecules comprises the target molecules exiting the first reservoir, entering the lumen part of the pore via the first opening, passing through the pore and exiting through the second opening of the aperture part into the second reservoir or vice versa, and wherein the electrolyte in the lumen has a resistance R_{L} and the electrolyte in the aperture has a resistance R_{A}, such that modulation of the resistance R_{A} occurs when a target molecule translocates through the aperture, and further such that substantially no modulation of the resistance R_{L} occurs when a target molecule translocates through the lumen.

29. (canceled)

30. (canceled)
